(19) United States
(12) Patent Application Publication
(10) Pub. No.: US 2023/0158261 A1
(43) Pub. Date: May 25, 2023

(54) SYSTEMS AND METHODS FOR BREATH DETECTION

(71) Applicant: Third Pole, Inc., Waltham, MA (US)

(72) Inventors: Fernando Trias, Newton, MA (US); Wolfgang Scholz, Beverly, MA (US); Gregory W. Hall, Belmont, MA (US)

(73) Assignee: Third Pole, Inc., Waltham, MA (US)

(21) Appl. No.: 18/057,145

(22) Filed: Nov. 18, 2022

Related U.S. Application Data
(60) Provisional application No. 63/264,336, filed on Nov. 19, 2021.

(51) Int. Cl. A61M 16/00; A61M 16/12

(52) U.S. Cl. CPC A61M 16/024; A61M 16/12; A61M 2230/63; A61M 2230/42; A61M 2016/0018; A61M 2202/0275

(57) ABSTRACT

Systems and methods are provided related to signal conditioning and analysis methods for detecting respiratory events of a human or an animal. Respiratory events detected can either serve as input to a drug delivery system or be a stand-alone breath detection device. Various methods for sensing respiratory events, processing respiratory signals, and analyzing respiratory signals are provided with the goal of enabling accurate and reliable detection of specific types of events in a respiratory cycle.

### RELATED APPLICATIONS

This application claims the benefit of and priority to U.S. Provisional Application Ser. No. 63/264,336, filed on Nov. 19, 2021, which is incorporated herein by reference in its entirety.

### FIELD

The present disclosure is related to systems and methods for breath detection of a human, mammal or an animal.

### BACKGROUND

There are a variety of techniques available to treat respiratory infections or other ailments of the respiratory system. For example, various medications can be delivered, including nitric oxide (NO). When treating the lungs or other parts of the respiratory system with a drug treatment, it can become important to deliver the treatment doses at the proper time and/or at proper intervals during the respiratory cycle of the patient.

### SUMMARY

The present disclosure is directed to systems, methods and devices for breath detection. In some embodiments, a breath detection system comprises one or more sensors configured to monitor at least one of one or more patient parameters and one or more environmental conditions relating to respiration, and a processor in communication with the one or more sensors. The processor is configured to analyze information from the one or more sensors, and determine an occurrence of at least one respiratory event using a threshold for detecting the at least one respiratory event, the threshold being variable throughout at least one respiration cycle to decrease the potential for a false determination of at least one subsequent respiratory event.

In some embodiments, the environmental conditions include at least one of ambient pressure, sound level and carbon dioxide levels.

In some embodiments, the processor is configured to vary the threshold using the information from the one or more sensors. In some embodiments, the processor is configured to vary the threshold based on an elapsed time with respect to a current detected respiratory event. In some embodiments, the elapsed time being based on the timing of prior respiratory events. In some embodiments, the elapsed time relates to a rate of breathing. In some embodiments, where the elapsed time relates to a duration of inhalation.

In some embodiments, the processor is configured to vary the threshold based on a current respiratory rate. In some embodiments, the processor is configured to vary the threshold for a period of time after a detected respiratory event, an amount of time of the variation of the threshold being based on the current respiratory rate. In some embodiments, the processor is configured to vary the threshold based on a duration of a prior inhalation. In some embodiments, the processor is configured to vary the threshold based on the duration of a drug delivery pulse. In some embodiments, the processor is configured to vary the threshold based on environmental conditions communicated to the processor using one or more environmental sensors.

In some embodiments, the processor is configured to vary the threshold based on patient activity communicated to the processor using a patient motion detector such that the processor is configured to vary the threshold based on a detected increase or decrease in patient activity. In some embodiments, the patient motion detector is an accelerometer.

In some embodiments, a breath detection system is provided that comprises one or more sensors configured to monitor at least one of one or more patient parameters and one or more environmental conditions relating to respiration, and a processor in communication with the one or more sensors. The processor configured to analyze information from the one or more sensors, and determine an occurrence of a respiratory event requiring drug delivery, the respiratory event requiring drug delivery if measurements associated with the respiratory event equal or cross a threshold value for detecting the respiratory event, the threshold value being variable.

In some embodiments, the techniques described herein relate to a breath detection system, wherein the drug delivery includes at least one dose of nitric oxide (NO). In some embodiments, the techniques described herein relate to a breath detection system, further including a NO generation system configured to produce the NO for the drug delivery.

In some embodiments, a method is provided that comprises monitoring, by at least one processor, a respiration associated with a patient, the monitoring of the respiration including receiving at least one respiratory measurement value measured by at least one sensor of a breath detection system, determining, by the at least one processor, a respiratory event requiring drug delivery based at least in part on the at least one respiratory measurement value, the respiratory event requiring drug delivery if measurements associated with the respiratory event equal or cross a threshold value, and varying, by the at least one processor, the threshold value associated with detection of the respiratory event based on conditions associated with at least one of patient parameters, environmental conditions, a respiration state, and respiration parameters.

In some embodiments, a drug for the drug delivery is nitric oxide (NO). In some embodiments, the method further includes communicating, by the at least one processor, with a further processor associated with a nitric oxide (NO) generation system for providing NO, a timing of the delivery of NO from the NO generation system relating to the detection of the respiratory event. In some embodiments, the method further includes instructing, by the at least one processor, a nitric oxide (NO) generation system to generate and deliver NO based on the determined respiratory event.

In some embodiments, the method further includes determining, by the at least one processor, timing of changing the variable threshold based at least in part on a prior respiratory duration.

In some embodiments, the measurement value crosses a threshold for a minimum amount of time before recording the respiratory event.

In some embodiments, varying the threshold value includes varying a magnitude of the threshold value.

In some embodiments, the method further includes determining, by the at least one processor, timing of changing of the variable threshold based at least in part on one or more of the timing and duration of a drug delivery pulse.

### BRIEF DESCRIPTION OF THE DRAWINGS

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1 depicts an exemplary breath detection system;

FIG. 2 depicts an embodiment of a breath detection system with a drug delivery system;

FIG. 3 depicts an example of drug delivery lagging by one respiratory period;

FIG. 4A depicts an exemplary breath analysis process;

FIG. 4B depicts an exemplary breath analysis process;

FIG. 4C depicts an exemplary embodiment of a breath analysis process utilizing more than one sensor;

FIG. 5 illustrates an exemplary graph showing an ILD respiratory waveform;

FIG. 6A depicts an exemplary breath detection method that utilizes more than one threshold;

FIG. 6B depicts an exemplary breath detection method that utilizes more than one threshold with the second threshold being dynamic;

FIG. 7A presents an exemplary graph relating to an approach where the respiration signal from a spontaneously breathing patient is inspiratory pressure;

FIG. 7B presents an exemplary graph relating to an approach where the respiration signal from a ventilated patient is inspiratory pressure;

FIG. 8 provides an exemplary graph relating to a data set with a moving average breath detection method;

FIG. 9A depicts an exemplary stochastic oscillator peak detection method;

FIG. 9B depicts another stochastic oscillator breath detection method;

FIG. 10 depicts an exemplary embodiment of a correlation breath detection approach;

FIG. 11A, FIG. 11B, and FIG. 11C depict an example of an FFT approach to breath detection;

FIGS. 12A, 12B, 12C, 12D, 12E, and 12F illustrates the principles of using an FFT with a Hanning window;

FIG. 13 depicts an exemplary flow chart for the process steps of the neural network to make a decision using a 2-dimensional input matrix;

FIG. 14 depicts an exemplary hidden Markov model that receives an input matrix of sensor data within a time window;

FIG. 15A depicts an exemplary basic breath detection state machine;

FIG. 15B depicts an exemplary breath signal and state machine steps;

FIG. 16 depicts representative breath profiles for various disease states;

FIG. 17 depicts an exemplary relationship between inspiratory duration (y axis) and breath period (x axis);

FIG. 18 depicts an exemplary respiratory cycle with the initial 60% of volume of the breath targeted for NO dosing;

FIG. 19 depicts an example of using a trapezoidal model of an inspiratory profile to estimate the time point coinciding with an inspiratory volume limit;

FIG. 20 depicts an instrumented mask for characterization of patient respiration;

FIG. 21A depicts an example of data from collection of cannula pressure and mask flow data;

FIG. 21B depicts the relationship that the controller can generate to assist utilizing cannula pressure as a proxy for inspiratory flow rate;

FIG. 22A depicts an embodiment where the window is located over a point of stability in the respiratory signal;

FIG. 22B depicts and embodiment where the inflection point is detected within a window of respiratory signal values;

FIG. 23 depicts a drug delivery system in operation with flow rates on the Y axis and time on the X axis;

FIG. 24 depicts a system that uses remote carbon dioxide measurement;

FIG. 25 depicts exemplary carbon dioxide measurements from a patient airway during breathing;

FIG. 26 depicts exemplary respiratory sensor data with a cough artifact;

FIG. 27 depicts exemplary respiratory sensor data with talking artifact;

FIG. 28 depicts an example of long cannula prongs;

FIG. 29 depicts an exemplary sweep of pulse duration to determine optimum treatment parameters;

FIG. 30 illustrates an exemplary embodiment of a NO generation system;

FIG. 31 depicts an embodiment of a NO generation and delivery system with a recirculation architecture;

FIG. 32 depicts a pulsed NO delivery system with pressurized scrubber and pressurized bypass architecture;

FIG. 33 depict embodiments of a NO generation and delivery system;

FIG. 34 depicts an embodiment of an exemplary graph showing an analog threshold-based event detection system utilizing a variable threshold;

FIG. 35A and FIG. 35B depict an exemplary graph of an analog event detection system utilizing a gating time window; and

FIG. 36 an exemplary electrical circuit for analog respiratory event detection.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following description provides exemplary embodiments only, and is not intended to limit the scope, applicability, or configuration of the disclosure. Rather, the following description of the exemplary embodiments will provide those skilled in the art with an enabling description for implementing one or more exemplary embodiments. It will be understood that various changes may be made in the function and arrangement of elements without departing from the spirit and scope of the presently disclosed embodiments. For example, breath detection through a nasal cannula is presented in many of the examples. The concepts presented herein are equally applicable to other gas delivery devices that can be utilized with an appropriate sensor to detect one or more inspiratory events including but not limited to nasal masks, face masks, tracheotomy tubes, ET tubes, manual resuscitation bags, CPAP machines, inhalers, and ventilators.

Specific details are given in the following description to provide a thorough understanding of the embodiments. However, it will be understood by one of ordinary skill in the art that the embodiments may be practiced without these specific details. For example, systems, processes, and other elements in the presently disclosed embodiments may be shown as components in block diagram form in order not to obscure the embodiments in unnecessary detail. In other instances, well-known processes, structures, and techniques may be shown without unnecessary detail in order to avoid obscuring the embodiments. It should be understood that many embodiments and solution elements can be implemented in either hardware, software or a combination of the two (e.g., signal filtering, comparison of values, thresholds, logic).

Also, it is noted that individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process may be terminated when its operations are completed but could have additional steps not discussed or included in a figure. Furthermore, not all operations in any particularly described process may occur in all embodiments. A process may correspond to a method, a function, a procedure, a subroutine, a subprogram, etc. When a process corresponds to a function, its termination corresponds to a return of the function to the calling function or the main function.

Subject matter will now be described more fully with reference to the accompanying drawings, which form a part hereof, and which show, by way of illustration, specific example aspects and embodiments of the present disclosure. Subject matter may, however, be embodied in a variety of different forms and, therefore, covered or claimed subject matter is intended to be construed as not being limited to any example embodiments set forth herein; example embodiments are provided merely to be illustrative. The following detailed description is, therefore, not intended to be taken in a limiting sense.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure relates to a respiratory device that is configured to detect respiratory events from one or more respiratory sensor signals. Examples of respiratory events include but are not limited to onset of inspiration, timing of peak inspiratory flow, end of inspiration/inhalation, beginning of exhalation, peak expiratory flow, and end of exhalation. Examples of respiratory signals include but are not limited to gas pressure, gas temperature, gas humidity, gas CO2 content, sound, gas mass flow rate, gas velocity, electromyogram (e.g. diaphragm EMG), transthoracic chest impedance, and EKG. The timing of respiratory events may be used one at a time (e.g., instantaneous flow rate) or in concert (e.g. average breath rate, duration of inspiration).

This disclosure is applicable to patient monitoring and inhaled drug delivery devices that are utilized to sense events within a respiratory signal (e.g., NO delivery, oxygen delivery, etc.).

In some embodiments, a breath detection system is a stand-alone device that can be integrated into other types of devices.

In some embodiments, false positives are prevented by analyzing a window of data instead of detecting the threshold crossing of a single value.

Some embodiments utilize a state machine approach to monitoring the state of the respiratory cycle. This can be utilized, for example, to define when to have breath detect active, identify respiratory events, and select respiration analysis parameters.

Some embodiments pertain to detection of false negatives. If a valid exhale event is not detected after an inhale, the system suspects that there is interference with the inspiratory signal (e.g., O2 concentrator in auto-pulse mode, patient motion artifact, cannula kinking). The delivery system stops sending medicine (e.g., inhaled NO) and waits for a valid inhale followed by a valid exhale to ensure that the breath detection system is functioning as intended. In some embodiments, inhalation detection is not armed until a valid exhalation detection event has been detected.

In some applications, inspiration detection has a variable delay determined by the stage of breath. It could be a binary on/off, or it could be based on a threshold of confidence (e.g., weighting pressure signal trigger threshold based on current inspiratory cycle state).

In some applications, the inspiration detection feature is turned OFF when a drug is being delivered.

Detection of patient respiration is a common need in the medical field. Whether it is for counting respirations or triggering the delivery of a drug, breath detection serves an important medical need but also presents many engineering challenges. Patient respirations are irregular at times and inhalation can be through the mouth, the nose, or both. Furthermore, patients can sneeze, cough, talk, snore, and generate motion artifacts that can obscure the underlying respiration signal.

In some embodiments, respiration signals are measured proximal to the patient, such as at the patient nose or diaphragm. In some embodiments, respiration can be sensed remotely with a respiration pressure signal traveling through a tube or cannula. In some embodiments, a respiration sensor is located on or near a patient. Respiration data or signal from the sensor can be transmitted to a breath detection controller via wired or wireless means.

Herein, the term “controller” may include any suitable computational hardware and/or software configured to control one or more additional components, sensors and/or actuators.

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

Various approaches are presented herein to analyze a respiration signal. These methods may be used individually or in one or more combinations.

Breath Detection Systems

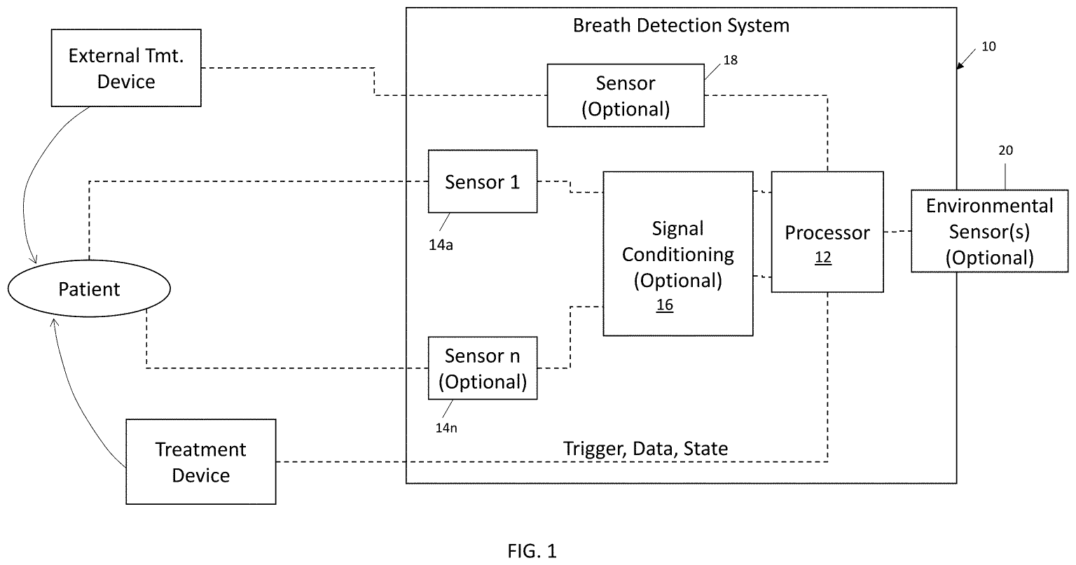

A breath detection system is utilized to detect respiratory events. These systems are typically part of a larger system that utilizes the detected breath information (e.g., monitor patient well-being, deliver drug at particular time points in the respiratory cycle, etc.). FIG. 1 depicts a block diagram of an exemplary breath detection system. The breath detection system 10 includes a processor 12 for collecting information and determining when a respiratory event occurs. The processor 12 collects data from one or more sensors 14a, 14n that monitor one or more patient parameters. In some embodiments, sensor data is conditioned (e.g., filtered, digitized, etc.) using a signal conditioner 16 prior to being read by the processor. Activity of an external treatment device can also be monitored by the processor with either direct communication to an external device or via an optional sensor 18. In some embodiments, the processor also collects information about the use environment using one or more environmental sensors 20 (e.g. barometric pressure, ambient acoustic noise, etc.) that can be used to one or more of 1) compensate or clean-up sensor data to remove artifact and other shifts and perturbations caused by ambient conditions, and 2) adjust respiratory event detection criteria in response to ambient conditions. The processor uses one or more algorithms (i.e., respiratory event detection processes or breath detection processes) to decipher respiratory events from the incoming sensor information. In some embodiments, the functions of the processor are reduced to practice as hardware (e.g. resistor-capacitor filters, amplifiers, logic gates, relays, etc.) to analyze one or more respiratory sensor signals and generate a trigger signal without utilizing software.

In some embodiments, the processor 12 may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

In some embodiments, the breath detection system is a stand-alone system with its own power supply, user interface, and data storage. In some embodiments, the breath detection system is embedded into another device (e.g. patient monitor, drug delivery device). In some embodiments, the breath detection system outputs information (e.g. trigger data, sensor data, respiratory state information) to a patient treatment device.

In some embodiments, the data storage may include, e.g., a suitable memory or storage solutions for maintaining electronic data representing the activity histories for each account. For example, the data storage may include database technology such as, e.g., a centralized or distributed database, cloud storage platform, decentralized system, server or server system, among other storage systems. In some embodiments, the data storage may, additionally or alternatively, include one or more data storage devices such as, e.g., a hard drive, solid-state drive, flash drive, or other suitable storage device. In some embodiments, the data storage may, additionally or alternatively, include one or more temporary storage devices such as, e.g., a random-access memory, cache, buffer, or other suitable memory device, or any other data storage solution and combinations thereof.

Breath Detection and No Generation/Delivery

A breath detection system can work in conjunction with a system configured to generate and/or deliver nitric oxide (NO). As explained above, a breath detection system can produce a triggering signal related to one or more respiratory events, such as inhalation and/or exhalation of a breath. This triggering signal can be produced by a processor associated with the breath detection system which can communicate with one or more sensors configured to collect information relating to the respiratory events. The triggering signal can be communicated to a drug delivery systems (i.e., a NO generation/delivery system), which can use the triggering signal to generate desired doses or concentrations of NO, and/or to deliver a desired amount of NO to a patient. In some embodiments, a common processor manages breath detection activities, drug generation activities (as applicable) and drug delivery activities.

In some embodiments, the system can be designed to detect a single type of respiratory event (e.g., an asthma attack, cough, hyperventilation, etc.). In response, the system can cause the delivery of a single dose of drug to treat the issue, and/or signal an alarm.

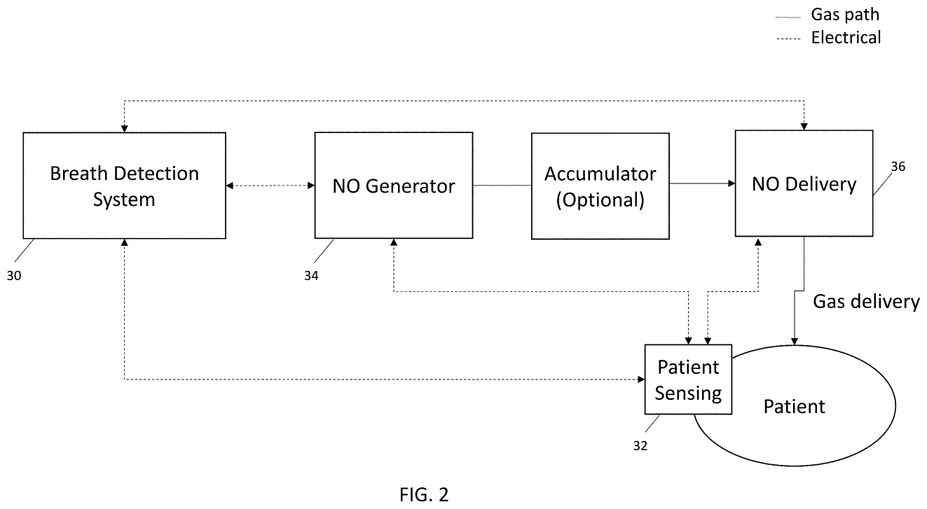

FIG. 2 depicts an exemplary embodiment of a breath detection system, NO generation system, NO delivery system, and patient sensing system. The breath detection system 30 receives information (dashed lines, e.g. sensor data, state information, dose settings, etc.) from the patient sensors 32, NO generation system 34, and the NO delivery system 36. In some embodiments, patient sensor information is passed through the NO generation and/or NO delivery system before reaching the breath detection system. In some embodiments, sensor signals are modified (e.g. filtered, DC shifted, etc.) or interpreted (e.g. determining patient state) before being sent to the breath detection system. It should be noted that modification of said signals can be done by hardware, software or both, depending on the implementation. The breath detection system receives external information and uses it to determine when one or more particular types of respiratory event occur (e.g. beginning of inspiration, end of inspiration, end of exhalation). These respiratory events are then communicated to other parts of the system as trigger signals, values (e.g. respiratory rate), or states to other parts of the system.

The breath detection system communicates with the NO generation system. In some embodiments, NO generation is continuous while other embodiments generate NO intermittently. In some embodiments, NO generation is continuous and at a constant level. The quantity of NO to generate is related to output from the breath detection system in some embodiments. For example, when the breath detection system detects a rapid respiratory rate (i.e. exceeding a predetermined threshold), the NO generator can increase NO generation to meet increased demand from the patient.

The breath detection system communicates with the NO delivery subsystem as well. For example, when an inspiratory event has been detected, a trigger signal is delivered to the NO delivery system to initiate release of NO to the patient. In some embodiments, NO delivery is intentionally delayed for an amount of time beyond the trigger point. The trigger signal from the breath detection system may be delayed by the breath detection system in some embodiments. In other embodiments, the delay is implemented by the NO delivery subsystem.

The NO delivery subsystem also sends information to the breath detection system in some embodiments. In one embodiment, the NO delivery subsystem communicates the actual timing of NO delivery to the breath detection system. This can be important since NO delivery can introduce artifact into patient sensor signals. By knowing the actual timing of drug delivery, the breath detection system can compensate for or ignore the artifact caused by delivery of NO and any purge gas as well.

Drug Delivery Lag

It should be noted that breath detection and drug delivery (e.g., NO) are related but not necessarily simultaneous. In some embodiments, when a breath is detected, the drug is released immediately. In some embodiments, additional decision-making occurs within the drug delivery system controller before release of the drug. For a non-limiting example, the additional decision making can involve one or more of patient dose, patient activity level, and current dose run rate. This additional decision-making can take time for computations and generation/release of drug. In some embodiments, the drug dose for one breath is delivered in a subsequent breath to permit adequate computational and drug processing time, the net effect being that drug delivery is time shifted with respect to respirations.

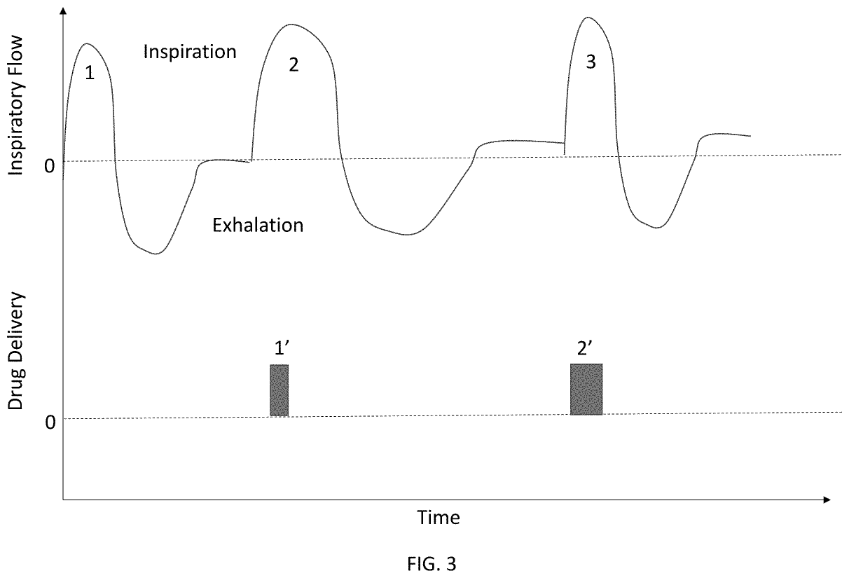

FIG. 3 depicts an exemplary graph showing the output of a breath detection and drug delivery system that delivers drug with a phase lag. In this example, the lag is one breath, however longer lag periods may be necessary due mechanical or computation time needs, for example. The system analyzes one or more initial breaths (one breath in this example) and calculates a dose for that breath. The system then delivers that dose to a second breath. The dose calculated during the second breath is delivered on the third breath, and so on. The amount of dose to deliver to a specific breath can be a function of one or more of respiratory rate, duration of one or more previous breaths, and prescribed dose level. In some embodiments, the amount of drug to deliver for the next breath is calculated based on an aggregation of the doses required for several prior breaths to smooth out the variance in dose delivery.

Respiration signals can be collected in a variety of ways from various types of sensors including but not limited to pressure (e.g., gas or surface contact), flow, strain, acceleration, sound, EMG, temperature, humidity, ultrasound, optical, and other types. In some embodiments, more than one respiration signal is collected and used as an input to breath detection. In some embodiments, the combination of two or more measurements are utilized to detect breath. In some embodiments, the device controller selects the strongest signal from a selection of two or more respiratory signals. For simplicity, most examples within this application describe pressure sensor signal analysis, however it should be understood that these other sensor measurements would be equally viable inputs.

The “breath detection” methods presented herein can be applied to the detection of many types of respiratory events, including but not limited to the beginning of inhalation, the point of peak inspiratory flow, the end of inspiration, the beginning of exhalation, the point of peak expiratory flow, the end of exhalation, the beginning of the pause between breaths, and other events.

It should be noted that an important aspect to any breath detection algorithm is the impact to the patient and surroundings of false positives and false negatives. An example of the problem with a false positive is triggering a drug delivery device to dose the patient when there was no inspiratory event. These types of errors can result in delivering drug when the patient is either not inspiring or at a stage of inspiration that is not desirable to dose. An example of a false negative is not detecting an inspiratory event when an actual inspiratory event occurred. False negatives can result in not dosing or underdosing a current breath and/or overdosing a later breath (as a means to catch up in the dosing run rate) in some systems. In one example, a breath detection system utilizes a pressure sensor measurement within the cannula as an input. The criteria for triggering inspiration is when the pressure in the cannula goes below a threshold for 25 milliseconds. During one breath, the pressure goes below the threshold for 24 milliseconds during an actual inspiratory event, which does not satisfy the conditions for an inspiratory event. This results in a false negative where the breath detection system does not generate a trigger even though an actual event occurred.

Continuing with the same exemplary system, the patient bumps into another person as they are walking, causing a rapid motion of the cannula and corresponding artifact in the cannula pressure signal. The pressure signal goes significantly below the pressure signal with the deviation lasting more than 25 millisecond. As a result, the breath detection system identifies the event as an inspiratory event and generates a trigger for drug delivery based on this false positive event.

Beyond false positives and negatives, other issues affect dosing. An example of dosing a patient when it is not desirable is providing nitric oxide to the approximately last ⅓ of inspiration in patients with chronic lung disease. Healthy lung is typically recruited first in these patients and the latter part of the breath fills lung tissue that is less effective at oxygen uptake. By dosing the latter part of the breath, unhealthy lung tissue is treated with nitric oxide, expanding the blood vessels in that locale and directing more blood to a region that has poor uptake. This can lead to a condition called “arteriovenous shunting” where blood passes through regions of the lungs without exchanging carbon dioxide for oxygen. One risk of false-positive inspiratory events is that a NO delivery system will deliver NO to the patient at a random time point within the respiratory cycle. In some instances, this random time point can coincide with the last ⅓ of the inspiratory event, thereby dosing, albeit unintentionally, unhealthy lung.

False positives can result in lower delivered dose and dose being delivered to the environment. This can present potential harm to both the patient and nearby people, respectively. In addition to the potentially deleterious effects of dosing at the incorrect time, false positive breath triggers can result in providing dose while the patient exhales. This results in underdosing the patient with the therapeutic drug and adding the drug to the patient environment. For example, a patient receiving oxygen that is dosed while they exhale will not receive any supplemental oxygen as if they weren't receiving treatment at all. In addition to underdosing, improper drug delivery timing results in wasted drug and potentially delivering drug to nearby people. In the case of inhaled NO, false positive drug delivery can lead to longer residence time of the NO with oxygen before inhalation leading to increased NO2 concentration in the inhaled gas.

False negatives also present a risk by underdosing the patient in the case of drug delivery or by underestimating respiratory rate in the case of breath counting. False negatives can result in lower patient dosing because an actual inspiration was skipped. This is particularly an issue with systems that dose the same amount of drug with every detected breath. This is less of an issue with systems that track a drug delivery run rate and simply add more drug to subsequent breaths when a breath is not detected.

In some embodiments, the risks associated with false positives and false negatives help guide the development of a breath detection algorithm to error on the side of patient safety. In some embodiments, patient safety means achieving the most accurate dose delivery. In some embodiments, patient safety means avoiding dosing a particular portion of the inhaled gas volume. In some embodiments, patient safety means minimizing the exposure of the patient to harmful byproducts (e.g., NO2).

Breath detection algorithms require an input signal or data stream. The data stream can be analog, digital, or a combination of the two. The input signal or data stream can be electronic, radio wave, optical, acoustic, hydraulic, thermal or other communication medium. It should be understood that various degrees of signal conditioning can be applied to the signals mentioned herein. For example, respiration signals may pass through filters (e.g., Butterworth) or moving averages (e.g., arithmetic mean, exponential moving average, weighted moving average, etc.) prior to being utilized in an algorithm. In some embodiments, a moving weighted average is calculated as the average of four recent measurements with the 1st, 2nd, 3rd, and 4th most recent measurements having weighting factors of 10%, 20%, 30% and 40%, respectively.

Concomitant therapies affect a respiration sensor signal and introduce additional signal content (e.g. offsets, frequency content, spikes, etc.). One example is an offset to the baseline pressure from a constant flow oxygen concentrator. Another example is a sound level added by a vibrating mesh nebulizer. In some embodiments, a breath detection algorithm receives a signal from a concomitant treatment device (directly or indirectly through a sensor) and compensates for the effects that signal may have on detecting respiratory events in one or more respiration signals. In some embodiments, a user can notify the breath detection system of concomitant therapies that are in use so that their influence on the respiration signal can be compensated for. In other embodiments, a concomitant treatment device communicates directly with a breath detection system to inform the breath detection system of its presence and/or activity.

Patient talking can also introduce variation into the respiratory signal. In some embodiments, a breath detection system utilizes a sensor (e.g., microphone, pressure sensor, piezo accelerometer, etc.) to detect when a patient is talking and modify the detection parameters, accordingly. Detection parameters modified by the controller in response to detected patient and/or environmental stimuli can include sensor sensitivity, delay settings, and current state (e.g., change to talking state). This approach can be applied to patient snoring, listening to music, coughing, sighing, eating, sneezing, eating, chewing gum or any other patient activity that is not a breath but can affect the respiratory signal.

Respiratory Method Identification

Information from the various sensors can be used by a breath detection system to determine the method of respiration being used by a patient. For example, inspiratory pressure signals vary, depending on the method utilized to deliver fresh oxygen-containing gas to the lungs. A patient breathing on their own, contracts their diaphragm to generate a vacuum within the lung and airway that draws air into the lung. As they exhale, the diaphragm relaxes and air is pushed out of the lung. When pressure within or near the patient airway is measured, inspiration is associated with negative gauge pressures and exhalation is associated with positive gauge pressure.

When a patient receives respiratory assistance from a ventilator, the ventilator delivers gas to the patient by generating positive pressure at the nose and/or mouth of the patient. Exhalation in this mode involves drawing a vacuum at the nose/mouth of the patient to pull gas out of the patient. Hence, the respiratory signal of a patient receiving respiratory assistance can be the inverse of the signal from a patient that breathes on their own.

A patient receiving respiratory assistance in the form of continuous positive airway pressure (CPAP) has a baseline pressure above atmospheric pressure. This elevated pressure helps hold the airway and lung open between breaths for maximal lung requirements and prevention of obstruction.

Some embodiments of a breath detection system function equivalently regardless of the polarity of the respiration signal(s). Other embodiments, such as those with fixed thresholds can be affected by changes in the respiration mode. For example, if a patient changes from breathing at atmospheric pressure to initiating CPAP, some embodiments of a breath detection system can detect the change in respiration mode and respond accordingly. In some embodiments, the breath detection system automatically stops treatment and sounds an alarm. In some embodiments, the breath detection system resets one or more parameters (e.g. the zero point) in order to accommodate the new respiratory mode and continues treatment. In some embodiments, a user or care giver will enter the respiratory assistance type (if any) into the breath detection system software. In some embodiments, a breath detection system can automatically detect the type of respiration.

Breath Detection Approaches

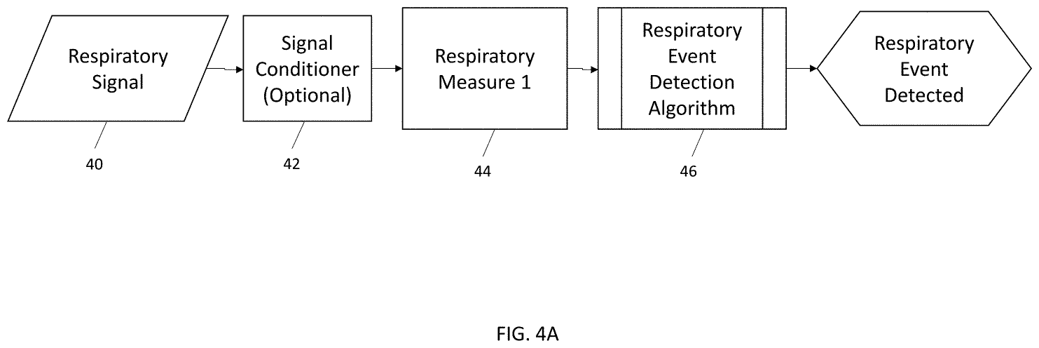

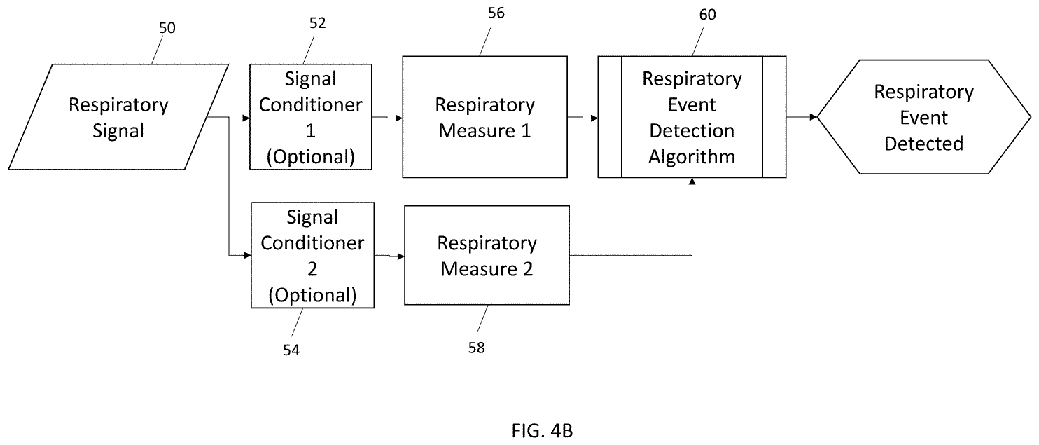

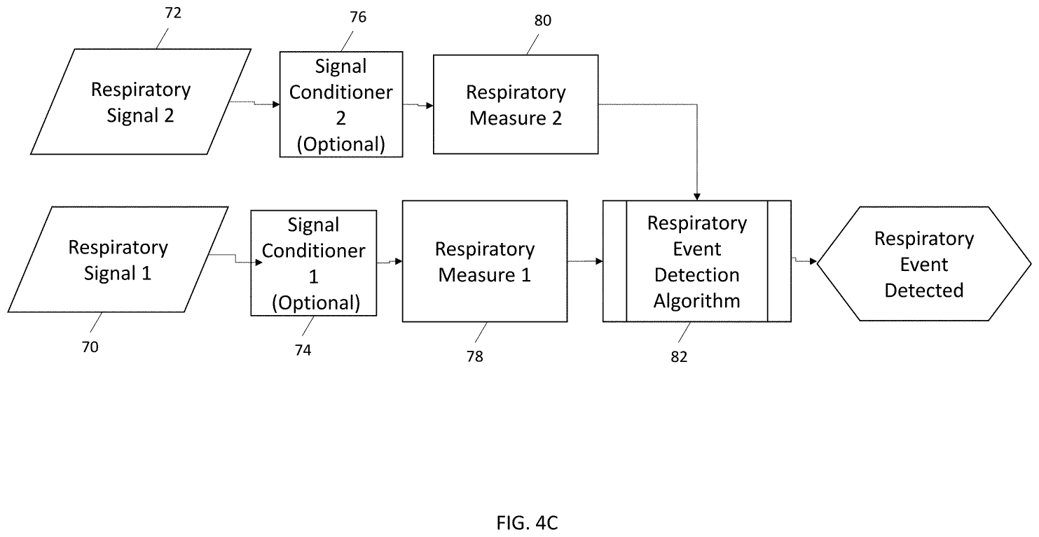

FIG. 4A depicts an exemplary breath analysis process. A respiratory signal 40 is collected and passes through an optional filter. In some embodiments, a signal conditioner 42 is utilized to precondition the incoming data. In some embodiments, the signal conditioner includes a filter utilized to one or more of remove artifact, smooth the signal and restrict the signal content to a specific range of frequencies. In some embodiments, the signal conditioner can be a filter, such as a low-pass or similar filter type used in speech processing and remove patient speech signals contained within the respiratory signal before the respiratory measure block. The filter may also remove other types of ambient noise contained in the respiratory signal, such as loud music or other sounds. In some embodiment, a low-pass filter with a cutoff frequency of 4.8 Hz is used, however other cutoff frequencies also may be used (e.g., in respiratory measure block 2 in FIG. 4B). In some embodiments, the signal conditioner amplifies one or more incoming signals. In another embodiment, the signal conditioner normalizes incoming signals or applies a DC shift to the incoming signal to a standard reference level. In some embodiments, the DC shift is equal to the respiratory signal baseline (during periods outside of inspiration/exhalation and may be a bias input to the signal amplifier. The output of the signal conditioner is a “respiratory measure” 44 that serves as input into a respiratory event detection algorithm 46. Examples of measures include but are not limited to the raw value from the sensor, a filtered value from the sensor, an average value from the sensor, the quality of the fit to a line or curve, standard statistical measures of the sensor signal (e.g. standard deviation), maxima/minima of a sensor value, a rate of change of a sensor value (e.g. derivative with respect to time), or the output of a computational model.

The respiratory event detection algorithm analyzes the respiratory measure to determine whether or not a specific type of respiratory event has occurred. Example breath respiratory event detection algorithms include but are not limited to seeking specific characteristics, specific patterns and/or threshold crossing events. In some embodiments, when a respiratory event has been detected, the respiration event detection algorithm output informs a treatment controller of the detected event and the drug delivery treatment is adjusted accordingly. Example adjustments include but are not limited to triggering delivery, delaying delivery, skipping delivery, adjusting drug dose (function of volume, concentration, flow rate, flow duration) and other actions.

FIG. 4B depicts another exemplary breath analysis process. In this process, a respiration signal 50 is passed through two independent signal conditioning processes 52, 54, creating two independent respiration measures 56, 58. The two respiration measures serve as input into a respiratory event detection algorithm 60. In some embodiments, the respiratory event detection algorithm determines whether or not a target type of respiratory event has occurred based on comparing and/or aggregating the two respiration measures.

FIG. 4C depicts an exemplary embodiment of a breath analysis process utilizing more than one sensor. Each sensor signal 70, 72 is optionally conditioned 74, 76 prior to further analysis. The filtered respiratory signals serve as respiratory measures 78, 80 and input to a respiratory event detection algorithm 82. In some embodiments, the respiratory measures represent measurements of the same parameter with independent sensors and the results are averaged. In some embodiments, the respiratory measures are of different parameters and the respiratory event detection algorithm decision is based on a combination of the two respiratory measures and/or independent analysis of both respiratory measures.

Respiratory Signals and Conditioning

Respiratory Pressure Measurement

There are two primary types of pressure sensors: absolute (relative to pure vacuum) and differential (measures the difference between two pressure signals). Absolute pressure sensors are susceptible to issues related to zero point change. Differential pressure sensors can compensate for changes in environmental pressure when one of the inputs is local atmospheric pressure and the other input is respiratory pressure.

Sensor Selection

Breath detection usually occurs at a particular pressure level, however, sensors that are sensitive at this level might not survive in this application due to the elevated pressure associated with drug delivery pulses. In some applications, such as nitric oxide delivery, the corrosive nature of NO and NO2 can harm the pressure sensor as well. Because of these considerations, a sensor cannot be selected for its sensitivity and signal range alone. Hence, compromises may have to be made in sensitivity and signal range in order to utilize a sensor that is sufficiently robust. As a result, the signal of the selected sensor may not have sufficient magnitude to provide analog to digital conversion resolution. In some embodiments, the breath detection pressure signal is amplified and filtered prior to analog to digital conversion.

Filtering

Transducer signals can be influenced by a multitude of sources. Examples include 60 Hz noise from power supplies, electromagnetic interference, random errors from manufacturing imperfections, analog to digital conversion noise, device component activity (e.g., motor vibration and power ripples), and other sources. Patient activity and physiology can also influence transducer signals. For example, a patient walking can introduce motion artifact into a cannula pressure measurement, or footstep noise into a microphone measurement. In another example, the patient's heartbeat can influence respiratory pressures. The patient environment can also introduce interference due to wind and, nearby noise sources (e.g., machinery, people talking, air conditioning, etc.).

A breath detection system utilizes one or more particular frequency ranges within a respiration signal. Hence, some embodiments filter other frequency ranges out of the respiration signal to provide a clearer input to the breath detection system.

Various types of filters exist and can be deployed to varying degrees. Filtering can be achieved in hardware, software or both. Over-filtering can be computationally intensive and result in diminished and/or delayed signal. Under-filtering can result in a signal that still contains artifacts that adversely affect a breath detection algorithm. For example, a breath detection algorithm that relies on the slope of a respiratory signal to detect breath requires heavy filtering to remove all noise in order to prevent false positives. However, the heavy filtering required results in decreased sensitivity (e.g., small breaths not detected) and a delay in the signal resulting in more time being required for the signal to cross a threshold leading to longer breath detection time.

Examples of signal filters include exponential moving average, infinite impulse response IIR filter, Butterworth, Resistor-Capacitor (RC) filters, and a multitude of others.

Adaptive Filtering

In some embodiments, the filter type and/or filter parameters are altered during the breath detection process. These filters can block certain frequencies of data that are not needed by the breath detection process. The filters can be digital or analog. The parameters of the filters can be adjusted based on algorithmic input that are derived from the signal, state of the patient, environment, or state of the device. In some embodiments, the cut-off frequency or band-pass frequency can be adjusted to include or exclude certain frequencies. In some embodiments with filters that use windows, the size of the window can be adjusted. In some embodiments, such as when using a moving average as a filter, the size of the window, and/or the weighting parameters can be adjusted.

Determining a Zero Point

The zero point in an algorithm serves as a datum or reference point for other measurements. In some embodiments, the zero point is defined at a time point when the patient is neither inhaling nor exhaling. In some applications, the zero point of a pressure signal is atmospheric pressure. In other applications (e.g., concomitant O2 delivery), the zero point can be offset from atmospheric pressure (e.g., mask application, constant flow application, CPAP application). The zero point can vary over time due to environmental conditions (e.g., weather, wind, and/or altitude), sensor drift, and patient actions (e.g., removal of O2 therapy). Thus, an inhaled drug delivery device must be able to determine a zero point and refresh it. In some embodiments, the zero point is determined at a specified frequency. In some embodiments, the zero point is determined continuously. In some embodiments, the zero point is re-established when certain events are detected (e.g., signal unstable, breath detection algorithm missing too many breaths, detected changes in ambient conditions, detected changes in patient activity, detected changes in concomitant device activity, changes in treatment mode or dose level)

There are multiple ways to derive a zero point, including but not limited to the following:
Regression within a window where the slope is zero (i.e. within an acceptable tolerance of zero). In some embodiments, a high R² value (i.e. at or above a specific threshold) or low standard deviation (i.e. at or below a specific threshold) within the window is also required. This identifies a point of stability which can serve as the zero point. This approach can be utilized in a specific transition within the respiratory cycle, such as transitioning from exhalation to inhalation.
Compare a two or more moving averages of differing window width. If they are similar, the signal is stable and can be used as the zero point. In one example, when a 0.5 second moving average and a 1 second moving average are compared and are sufficiently similar (e.g. within 5% of each other), the zero point is recorded.
If the 1^{st} derivative of a signal is zero, the signal is either at a local maximum, local minimum, or a stability point. In some embodiments, the location within the cycle is identified by the magnitude of the signal and/or signal properties leading up to the stability point (e.g. rate of change with respect to time a set amount of time prior). In some embodiments, the location within the cycle is identified with a state machine approach.
   In some embodiments, a stable point relating to the end of inhalation cannot be identified because a patient transitions from inhalation to exhalation and back to inhalation too rapidly. In this case, the zero point can be calculated as a function of the maximum and/or minimum respiratory sensor values. In some embodiments, the zero point is the average of the maximum and minimum stability values. In some embodiments, the zero point is equal to the minimum stability value plus a fraction of the difference between the minimum and maximum stability values. In some embodiments, an average of 2 or more minimum stability values is utilized as the zero point.
Look at the beginning and end point of a window. If the values are the same for sufficient time, that is indicative of the zero point. In some embodiments, a window of 0.5 seconds is utilized and when the leading data point and trailing data point are within 5% of each other for 50 msec, a zero point can be recorded.
Look at three or more points in a moving window. If the points are more or less equal, then the data signal is stable within the window, which is indicative of the zero point. In some embodiments, data points are observed at the beginning, midpoint and end of a fixed-width (e.g., 0.5 second) moving window. In some embodiments, the data points are not evenly spaced within the window.

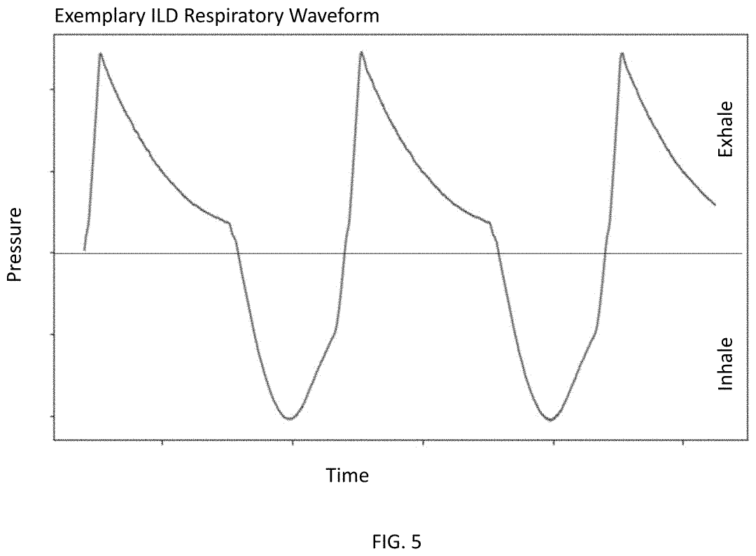

Patients with interstitial lung disease (ILD) present a challenge to determining a zero point because they immediately transition from exhalation to inhalation and from inhalation to exhalation, as shown in the exemplary graph of FIG. 5. In applications such as this where there is no quiescent period at the zero point, a zero point can be estimated as a function of the high and or the low values of the signal (e.g., average of high and low values, average of low value, most recent low value, etc.). It should be noted that in some embodiments, drug delivery interferes with the respiratory signal. In some embodiments, drug delivery is briefly paused so that continuous data for the entire respiratory cycle can be acquired to calculate the zero point from the high and low values prior to resuming drug delivery.

In some embodiments, an inhaled drug delivery device measures the zero point during each pause after exhalation in a respiratory cycle. This measured zero point can be used to update the zero-point used for detection over time. In some embodiments, the zero-point used for analysis is a function of previous zero-point measurements (e.g., a moving average of zero-point measurements, a probability function of the zero-point).

Respiratory Signal Analysis

In some embodiments, a breath detection algorithm receives various inputs from sensors, external devices, and/or users and generates a flag (i.e., a trigger event and/or a recorded data point) to mark a particular respiratory event. There are multiple approaches to breath detection that can be effective. Many approaches detect a point of instability or stability within a signal vs. a simple threshold-crossing or slope magnitude-based criterion. In some embodiments, a breath detection signal (e.g., triggering event message) is generated instantly when criteria are met. In some embodiments, criteria need to be met for a certain amount of time or quantity of data points prior to signaling the detected event. In some embodiments, more than one breath detection approach is utilized and the event is signaled only when there is a minimum amount of agreement between methods.

Filter to Threshold Comparison

Respiration signals can include considerable noise and artifact. In some embodiments, one or more respiration signals are filtered prior to comparison to a threshold indicative of the beginning of inspiration. An inspiration is marked when the filtered value or function of filtered values crosses a threshold in a particular direction.

Variable Threshold

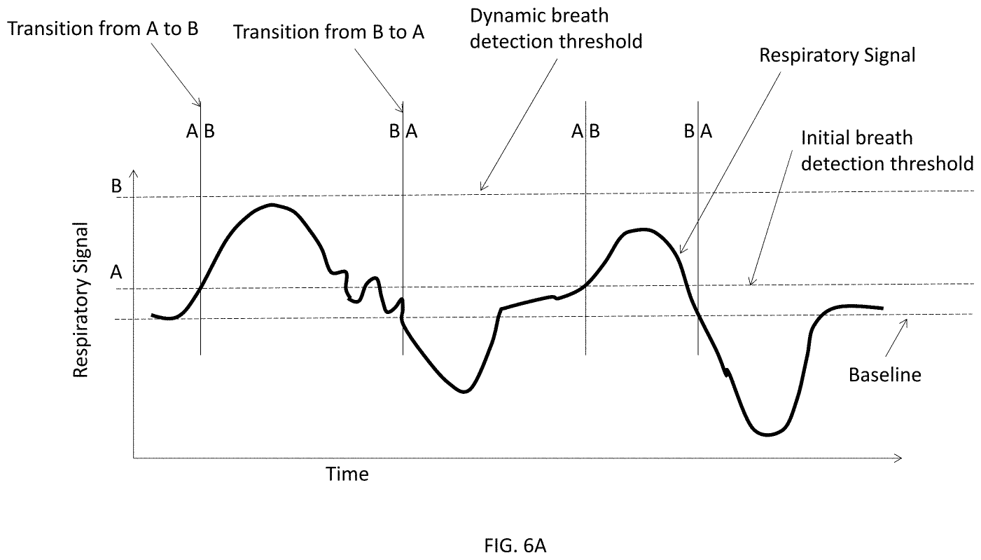

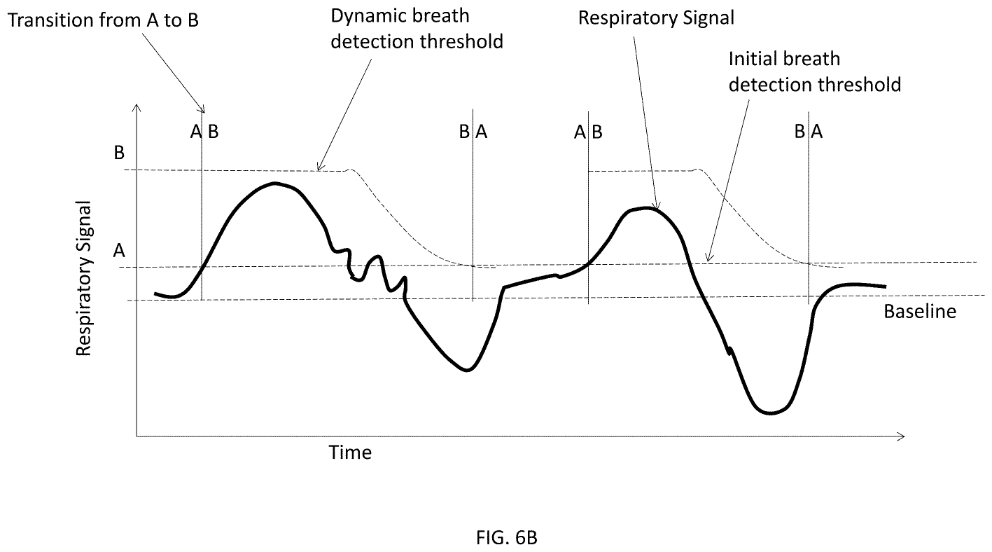

In some embodiments, the threshold for breath detection changes throughout the respiration cycle to decrease the potential for false positives. FIG. 6A depicts an exemplary graph showing a breath detection threshold of a respiratory signal (raw or filtered) having a value of “A” until the threshold is crossed. After the threshold “A” is crossed, the system changes the breath detection threshold to “B” to prevent false positives. In some embodiments, the breath detection threshold remains at “B” for a period time. This period of time is a fixed amount of time in some embodiments. In some embodiments, the period of time, itself, is dependent on other characteristics of the respiratory signal (e.g., until the respiratory signal falls below a threshold (e.g. Level A, or zero), or for a fixed percentage of the average of one or more prior breath periods). In some embodiments, the threshold transition from Level B back to Level A occurs after an event (e.g. exhalation detected) or after the respiratory signal has been below a threshold for a minimum amount of time. In the example in FIG. 6A, the inspiration detection threshold resets to A as a step function when the respiratory signal crosses the zero point and the patient is entering exhalation.

The breath detection threshold can then change again by step function, gradually, or some other function over time until it reaches a value of “A” again. In the embodiment shown in FIG. 6B, the threshold gradually decreases towards a value of “A” over time. In some embodiments, the breath detection threshold “A” is lowered in order to set an appropriate sensitivity of the system to current patient (e.g., when there has been a prolonged absence of breaths detected) and environmental conditions (e.g., ambient noise levels decrease) enabling a lower breath detection threshold “A.”

In some embodiments, a breath detection system determines a noise level for the sensor data by observing the values of sensor data during a period of stability. The system then uses these observations to calculate a minimum value that will be accepted by the system in the case where an algorithm uses a threshold. In the case of a dynamic threshold that changes over time, the dynamic threshold will not be allowed to fall below a proportion of the minimum value. This minimum value can also be used by algorithms that do not use a threshold in order to determine the validity of data or to adjust data by smoothing out the data to compensate for the noise level.

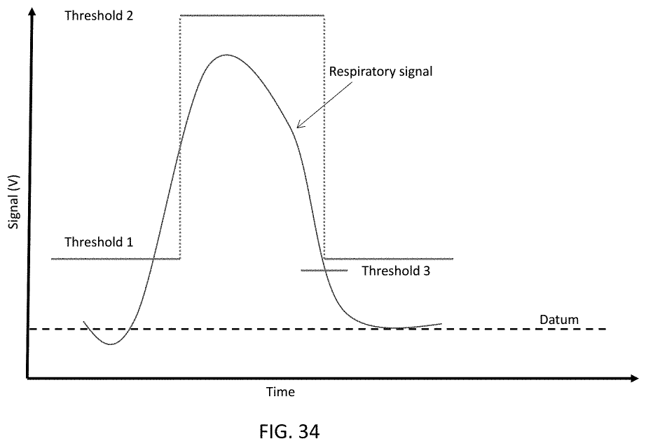

In some embodiments, electrical circuits (i.e. hardware) are utilized to monitor a respiratory signal and determine crossing of or more thresholds. FIG. 34 depicts an exemplary graph showing a threshold-based hardware system in which an analog voltage comparator indicates a detected respiratory event. When voltage from a pressure-based breath sensor exceeds the Threshold 1 reference voltage, a respiratory event is detected and the Threshold 2 reference voltage is enabled. Threshold 2 is typically set to a high value that will prevent false-triggering. A downward crossing at “Threshold 3” indicates the end of a breath and resets the system to utilizing Threshold 1.

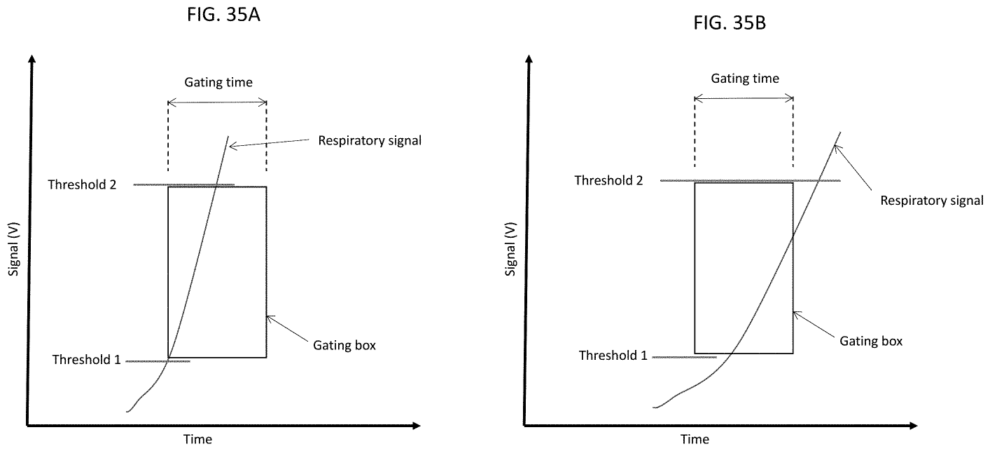

FIG. 35A depicts another exemplary graph of a hardware approach to respiratory event detection. A gating system uses rise-time (i.e. rate of change in a respiratory signal) as a basis for detecting breath. When a signal from a respiratory sensor crosses Threshold 1 value, the system monitors the signal for crossing a second reference voltage, Threshold 2, and a “gate timer.” The gate timer remains active for a preset time duration (gating time). When the gating time is over, the Threshold 2 reference voltage is disabled. This has the effect that only breath signals that have a sufficiently fast rise-time will indicate a breath detection. The gating time and Threshold 1 and Threshold 2 reference voltages form a box in voltage-time space. When the respiratory sensor signal exits the top of the box, as in FIG. 35A, the rate of change has been sufficient to register a respiratory event (e.g. inhalation). When the pressure signal exits the side of the box as depicted in FIG. 35B, no respiratory event is detected.

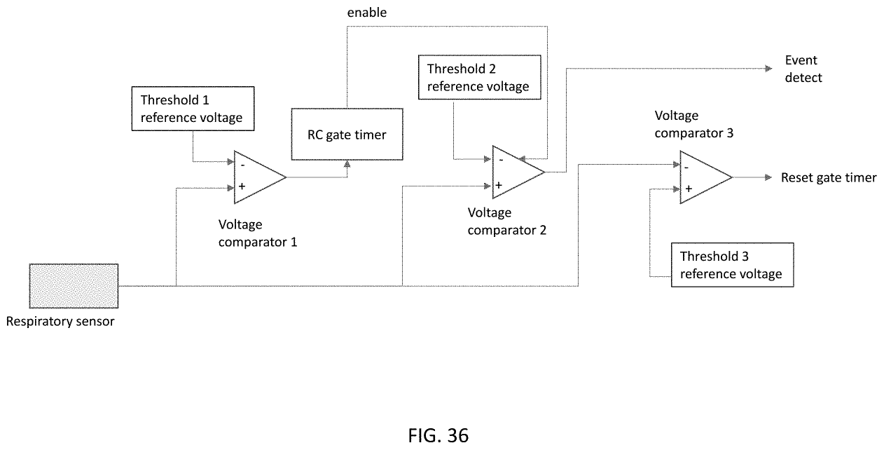

FIG. 36 depicts an embodiment of an electrical circuit for respiratory event detection utilizing an RC gate timer. At the input of the gate timer, an edge detector senses a voltage rise from the Threshold 1 voltage comparator. The timer then produces a single pulse of preset time width which then enables voltage comparator 2 (e.g. a tri-state comparator) for the preset amount of time. The output of the respiratory sensor must trigger voltage comparator 2 before the enable pulse terminates in order for a respiratory event to be detected.

Derivative Method

In some embodiments, a derivative (e.g., first derivative) of a respiration signal with respect to time is utilized to identify a breath. When the rate of change in the signal exceeds a threshold, a breath is detected. In some embodiments, when the rate of change in the signal exceeds a threshold for a specified amount of time, a breath is detected.

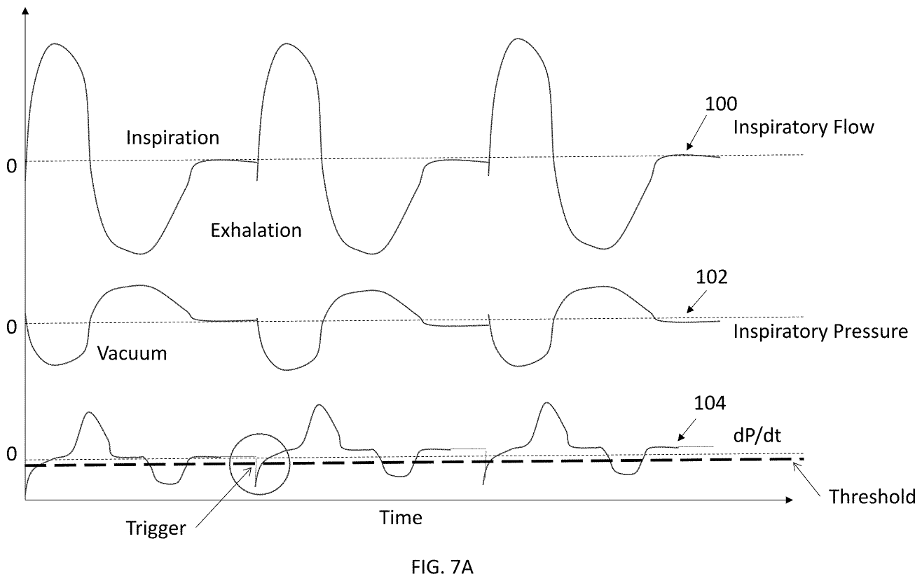

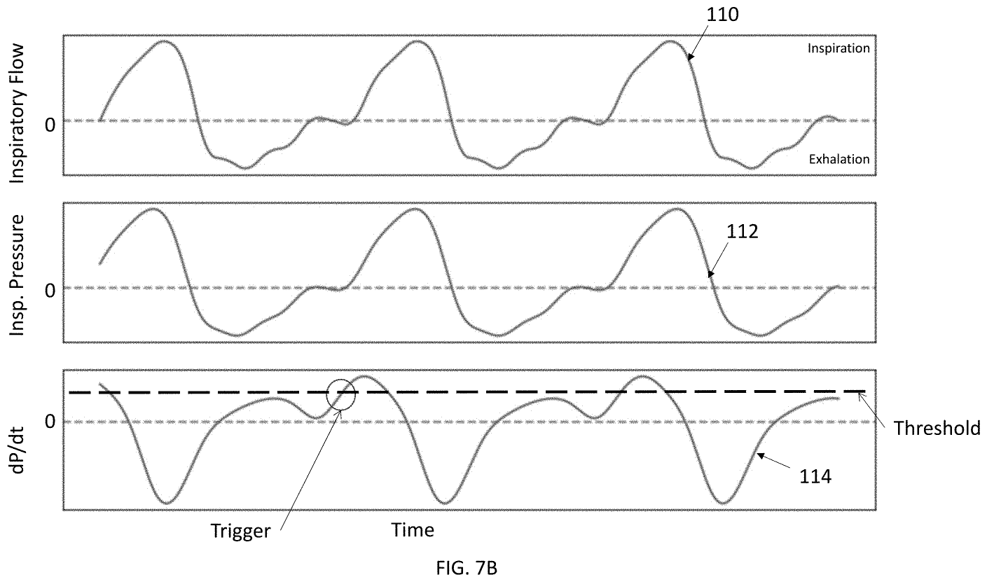

FIG. 7A illustrates an exemplary graph relating to this approach where the spontaneous respiration of a patient is monitored with an inspiratory pressure sensor. A curve 100 shows the inspiratory flow rate, where positive values indicate flow into the patient. A curve 102 depicts the inspiratory pressure with negative values indicating vacuum pressures (below ambient pressure). A curve 104 depicts the first derivative of the pressure signal in time. The spike in slope of the pressure signal provides a significant event that is easily detected with a computational approach to detect an inspiratory event.

FIG. 7B depicts exemplary graphs for the derivative approach for a patient that is breathing on a ventilator. A curve 110 depicts an inspiratory flow signal for a patient on a ventilator. Positive inspiratory flow is associated with inhalation and negative flow is associated with exhalation. A curve 112 depicts the inspiratory pressure. In this case, the ventilator generates the inspiratory flow and pressure increases as the patient inhales. A curve 114 depicts the derivative of pressure with respect to time. The rapid rise in pressure associated with inhalation manifests as a positive spike in the dP/dt curve, crossing a threshold and indicating that inhalation has begun. Although, the pressure signal for ventilator treatment versus spontaneous breathing can be inverted, the derivative approach functions in the same way to detect discontinuities in the inspiratory pressure signal.

Although the examples provided both utilize inspiratory pressure for the derivative method, it should be understood that the same approach can be utilized for a myriad of respiratory signals including but not limited to inspiratory flow, chest impedance, diaphragm EMG and other approaches. The derivative method can be effective on clean signals but can exacerbate noise in a signal, generating false positives. False positives can result in wasted drug when drug is released for a non-event, but also can impact a breath detection approach at a higher level, affecting processes that analyze a breathing signal (e.g., state machine approaches, pattern recognition approaches). In some embodiments, delivery of drug can interrupt the respiration sensor data stream.

Value to Filter Comparison

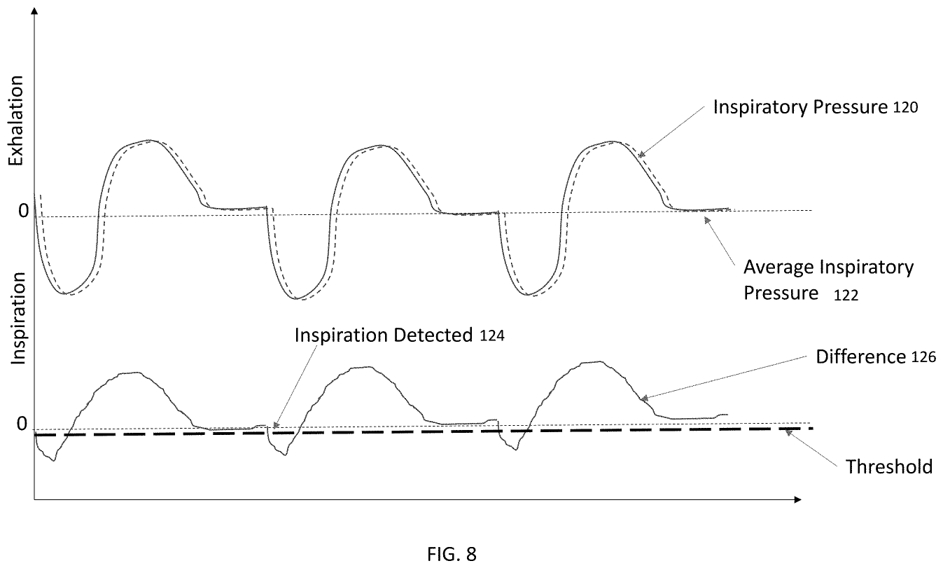

FIG. 8 provides an exemplary graph relating to a data set with a moving average breath detection. Curves 120, 122 depict pressure (solid line 120) and moving average (dashed line 122). Curve 124 depicts detected inspiration, and curve 126 shows the difference between the current measurement and the moving average (reading−average). When the difference crosses a threshold, breath is detected. In some embodiments, the pressure delta threshold is a fixed value in the range of 2 to 20 mbar. In some embodiments, the pressure delta threshold is continuously varied in response to environmental factors and properties of the respiratory signal. For example, in some embodiments the pressure delta threshold can be increased in the presence of ambient noise or when the patient is talking.

In some embodiments, breath detection is triggered when the pressure exceeds a threshold and the moving average is below a threshold (indicative of the pulmonary system in a quiescent state, like the end of exhalation or the pause after exhalation).

In some embodiments, the respiratory sensor signal is passed through two different filters. The difference between the two resulting signals can be used to detect a breath. In one exemplary embodiment, a 0.1 second moving average and a 0.5 second moving average are utilized.

Stochastic Oscillator

In some embodiments, an analysis technique similar to a stochastic oscillator is utilized to detect events in the respiratory cycle. This analysis may be calculated using the following equation:

$$Oscillator Value = \frac{C - {L}_{t}}{{H}_{t} - {L}_{t}}$$

where C is the current value, Lt is the lowest value during the previous time period (t), and Ht is the highest value during the previous time period (t). The value can be multiplied by 100 to be presented in terms of percent, if desired.

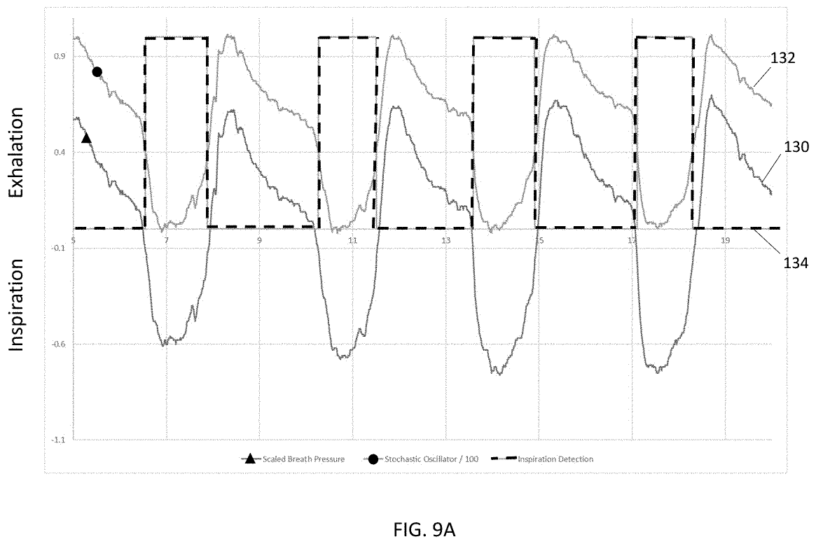

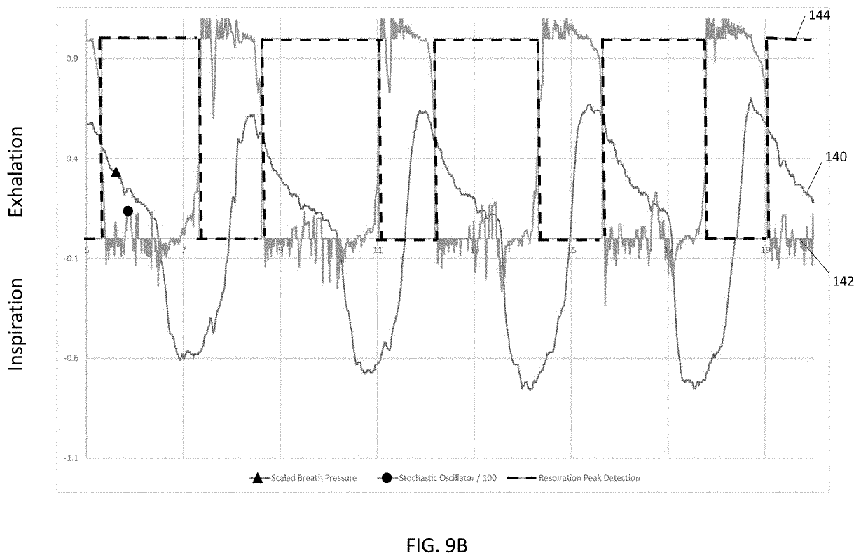

FIG. 9A depicts an exemplary graph showing a stochastic oscillator peak detection method where t is longer than the expected respiratory period (in this case 5 seconds), and the threshold is 0.4 (40%). The graph depicts scaled breath pressure curve 130, stochastic oscillator curve 132, and an inspiration detection curve 134. When the oscillator value exceeds a threshold (0.4), a start of inspiration event is marked and when it returns below the threshold, an end of inspiration event is marked.

In other stochastic oscillator embodiments, a longer period spanning multiple breaths is used. This approach can be more resistant to the effects of transients and noise, but less responsive to real changes in breathing pattern.

FIG. 9B depicts another stochastic oscillator breath detection method. The graph depicts scaled breath pressure curve 140, stochastic oscillator curve 142, and an inspiration detection curve 144. As shown, the threshold is used to detect the start and/or end of exhalation. The system uses a time period much shorter than the expected respiratory period, 0.25 seconds in this example. When the value exceeds a threshold (0.5 in this example), the timing of peak pressure of inspiration or expiration are marked. This example finds regions where the respiratory sensor signal changes directions, identifying local minima and maxima in the respiratory signal.

Correlation Models

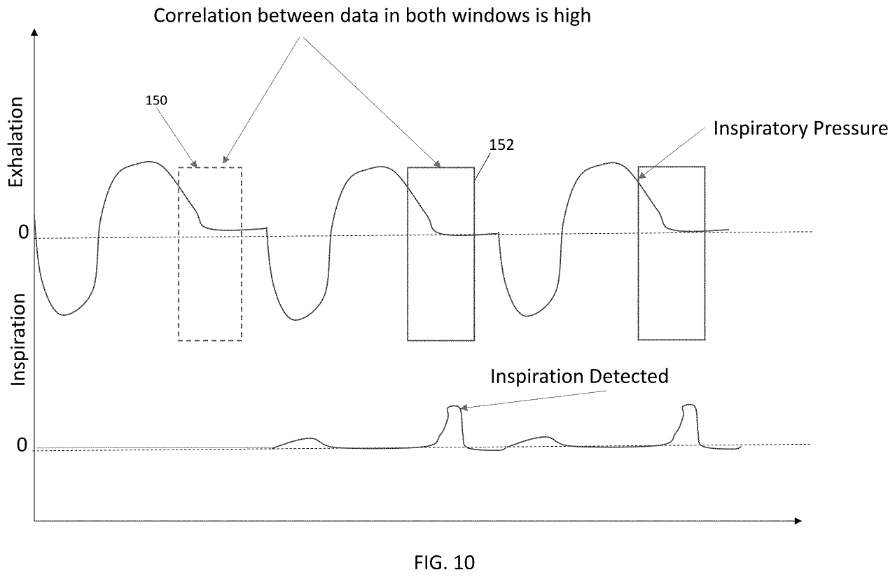

Correlation models can be utilized to mark the onset of inspiration. The correlation model utilizes a sliding of a window of past data to predict future data. FIG. 10 depicts an exemplary embodiment of a graph showing a correlation breath detection approach. A reference window 150 of data at a point relative to the start of inspiration is compared with data within a moving window 152, that is moving forward in time. In some embodiments, the width of the window is a fixed duration of time or data points. In some embodiments, the width of the window is varied based on patient conditions (e.g., respiratory rate) or patient activity (e.g., active vs. sedentary). When the moving window encounters a subsequent inspiratory event, the correlation value increases due to the similar curve shape. the respiratory event is marked when one or more of the following conditions occurs: the correlation value exceeds a threshold, and the correlation value reaches a local maximum. In some embodiments, the reference window data consists of a hypothetical/ideal curve shape, rather than actual patient data. In some embodiments, the representative curve shape is determined by the breath detection system from one or more of the hypothetical curve, environmental data, and patient data.

In some embodiments, the data sampling rate is varied with breath rate. The minimum usable sampling rate is roughly twice the respiratory rate. For example, a sampling rate can be roughly 10× the respiratory rate. Fast sampling rates are not necessary for correlation models since they are simply looking for patterns in the data. In some embodiments, the correlation value is the R2 value from a regression of the two data windows being compared. In some embodiments, a correlation coefficient is computed to compare (e.g., statistics of similarity: RMS, square of differences, Pearson correlation coefficient) the data windows. In one embodiment, the correlation value is determined by subtracting the array of values making up the current window from the array of values representing the reference window to make a difference array. When the sum of the difference array values or the average of the difference array values are below a acceptance criteria level, a target respiratory event has been detected. In some embodiments, the current data stream is compared to the average of two or more prior windows.

The breath detection system utilizes a moving window to detect breaths and qualifies them. In some embodiments, the cross-correlation is utilized as a qualifier for other methods (i.e., a quality check to confirm that breath prediction is likely). Correlation models can be utilized to detect any portion of a respiratory signal. For example, a correlation model can be utilized to detect the pause after exhalation, which, in turn, arms a threshold detection algorithm to detect inspiration. Correlation models can also be utilized to identify artifacts in the respiratory signal based on their characteristic waveform. For example, a sneeze or cough, have a unique signature that can be identified using a correlation model. In some embodiments, a correlation model can be compared to a hypothetical artifact source. In some embodiments, characteristic data are collected from the patient to be utilized as the reference signature.

Fast Fourier Transform (FFT)

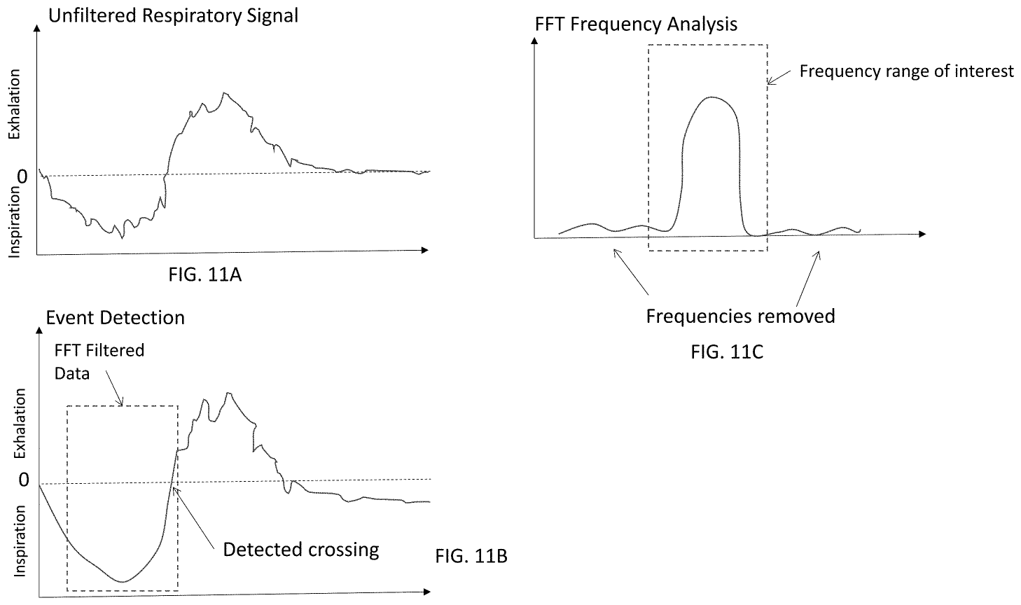

In some embodiments, the respiration signal is digitized in real time. As the data samples are captured, sliding overlapping buffers of these data are taken, and a smoothing window is applied to each window. The smoothing window can be of many types (like Hanning, Hamming, raised-cosine, etc.). A Fast Fourier Transform (FFT) is applied on each consecutive windowed buffer to transform the data from time domain to frequency domain. The resulting FFT data for each buffer can be further processed to keep the amplitude of desired respiration signals intact and suppress other frequencies. The desired respiration frequencies are typically dominant in frequency and can be determined from an average of multiple FFT data. If the interest is in breath rates between 8 and 40 breaths per minute, the amplitude of the frequencies that correspond to this breath rate range are kept intact and other frequency amplitudes are suppressed. Other methods can be used to suppress the noise outside the desired frequency band using different algorithms and windowing types. The processed FFT data window can be then transformed into the time domain to reconstruct the respiration signal while accounting for the overlap added to the original signal. As a result, the reconstructed respiration signal has reduced noise and improves the accuracy of the breath detection algorithm. Coughing, talking, snoring and other irregularities can be eliminated from the respiration signal using this technique. FIG. 11A, FIG. 11B, and FIG. 11C depict exemplary graphs of an FFT approach to breath detection.

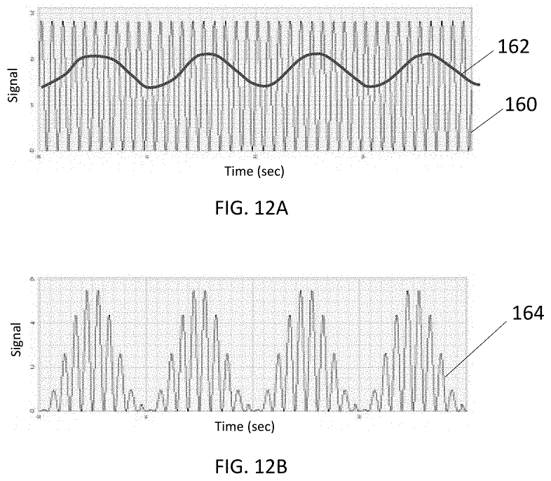

FIGS. 12A, 12B, 12C, 12D, 12E, and 12F illustrate exemplary graphs showing the principles of using an FFT with a Hanning window as an example of elimination of undesired frequency components to clean the signal spectrum and reconstructing the clean signal using an IFFT and accounting for overlapping windows. FIG. 12A depicts an exemplary graph of a clean respiratory signal 160 represented by a 10 Hz sine wave. An exemplary Hanning signal 162 with 1-second width is superimposed on the clean respiratory signal with 0% overlap between windows. FIG. 12B depicts the mathematical product of the respiratory signal and the 0% overlapping Hanning windows (curve 164). FIG. 12C depicts a 1 Hz Hanning signal 166 and a 10 Hz respiratory signal 168 with periodic 100 Hz noise occurring every 1 second. FIG. 12D depicts the same respiratory signal 168 with noise superimposed with two 1 Hz Hanning signals 170, 172 that are 50% overlapping in time. It should be noted that one Hanning signal has a value of zero when the noise occurs while the other Hanning signal has a maximum amplitude at the time of the noise. By overlapping Hanning signals, it can be assured that no important respiratory signal content is lost. FIG. 12E depicts the contents of the respiratory signal in the frequency domain for the combination of the two Hanning signals. A filter is applied to frequencies above 20 Hz in this example to eliminate noise frequency components that occurs at frequencies above a target range for respiration. In FIG. 12F, the frequency content below the filter cut-off are reconstructed into the time domain to create a clean respiratory signal 174 within the analysis window. When utilized in real time breath detection applications, multiple overlapping FFT analysis with 1 or more Hanning window or other type of windows can be occurring at a time with a new window established periodically (e.g., every 50 microseconds). This approach is computationally intensive, however manageable by modern microprocessors and specialized signal processing chips can handle this approach. This approach introduces a small amount of lag into the data analysis to clean the signal that can be acceptable for the application but improves the accuracy and speed for which a respiratory event can be detected (e.g., threshold crossing event), owing to the cleaned-up signal. To minimize the delay introduced by sliding overlapping windows and maximizing the integrity of the signal, the overlap between consecutive windows can be maximized, depending on the application, for a cleaner signal.

In another application of FFT, FFT is utilized to identify the primary frequency of a respiratory signal, i.e. the respiration frequency. Then, a Butterworth (or equivalent) band-pass filter centered on the respiratory frequency is utilized to remove artifact from the respiratory signal. This cleans the respiratory signal to improve the accuracy and reliability of respiratory event detection.

Neural Network

In some embodiments, a neural network method is utilized to detect inhalation. In some embodiments, the neural network consists of multiple connected layers. As input, a sliding window of data is provided (e.g., a matrix with n time steps as rows and m columns of sensor data from m sensors). This window includes data from one or more sensors for a period of time ending with the latest data collected. The matrix of data for each time step is manipulated by a sequence of transformations, AKA a neural network. The transformation sequence is developed by training using an annotated database of respiratory data where the annotations indicate whether or not there is an actual breath. As output, the neural network gives a likelihood that the window of data will be immediately followed by the start of a target inspiratory event and/or already includes a target inspiratory event.

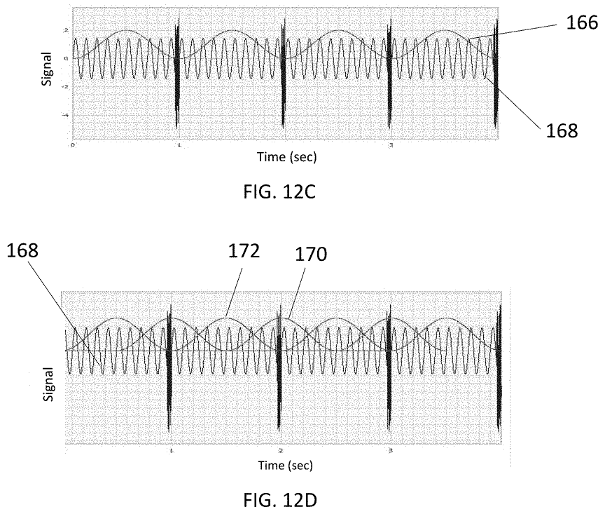

FIG. 13 depicts an exemplary flow chart for the process steps of a neural network to make a decision using a 2-dimensional input matrix. The exemplary input matrix 180 consists of sensor data in columns for a window of time with the matrix rows being each time step. This type of approach can accommodate data from multiple sensors, hence the “Sensor n” nomenclature shown in FIG. 13. It should be obvious to someone skilled in the art that the transposition of this exemplary matrix would be functionally equivalent. The same principle applies to using a multi-dimensional tensor as the input, an example of which would be two or more matrices at different time points.

First, the input is transformed by a transformation operation 182. Then, the output of that transformation serves as input to an activation operation 184. In some embodiments, the transformation operation is a matrix multiplication, and the activation operation is a non-linear function. In some embodiments, the non-linear function is a sigmoid function which takes the form of

$$S ( x ) = \frac{1}{1 + {e}^{- x}}$$

The combination of a transformation operation and an activation operation constitutes a “layer”. A complete neural network consists of one or more layers that results in an output. The output can be binary (e.g., inspiration, no inspiration), a scalar (e.g., the state of the respiratory cycle), a vector (e.g., a vector where each element is a probability of being in a particular respiratory cycle state), a matrix, or a tensor. Layers can be applied sequentially with the output of one layer serving as the input to a subsequent layer as shown in FIG. 13, or in parallel to form a network. The neural network can be used to recognize patterns in the respiratory signal (e.g., the time before inspiration, patient coughing).

A neural network can be pretrained and fine-tuned with an initial dataset. Pretraining involves subjecting the model to representative data (e.g. filtered data) to teach the algorithm general patterns. In some embodiments, the dataset is fictitious data designed to represent specific scenarios and the like. In some embodiments, the dataset is a collection of real-world data from exemplary patients. The intention of pretraining is to subject the model to a data set (preferably a large one) that introduces the general trends of the phenomenon to be analyzed. After pretraining, a neural network can be fine-tuned with a more focused set of data with relevance to a specific task (e.g. ILD patient data or data from a specific patient).

The initial dataset may include a set of input matrices, each input matrix being paired with a known output. The neural network may ingest each input matrix and generate a predicted output based on the layers of the neural network. An optimizer may then assess the predicted output to the known output for each input matrix to determine error and retrain the parameters of the layers of the neural network. In some embodiments, the optimizer may use a loss function to assess the error, such as, e.g., Hinge Loss, Multi-class SVM Loss, Cross Entropy Loss, Negative Log Likelihood, or other suitable loss function. The resulting error can be used to update the parameters by applying the loss to the neural network via a backpropagation technique, such as, e.g., gradient descent (e.g., stochastic gradient descent) or other backpropagation technique.

Markov Chain

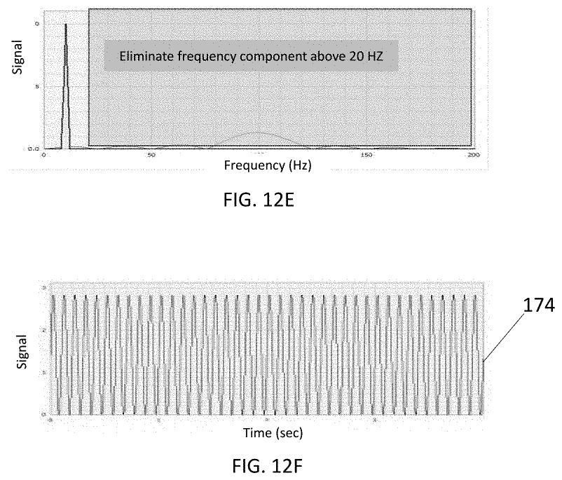

In some embodiments, a breath detection algorithm can employ Markov models. A Markov process consists of a list of states where each transition state has a calculated probability. These embodiments involve a Markov chain whereby the next value in a series depends on prior values. Similar to a state machine that depends on the conditions at that time (including prior data). Several output states are defined for the breath, including one or more of: the start of inhalation, end of drug delivery pulse, end of inhalation, start of exhalation, and end of exhalation. In some embodiments, the architecture and data manipulation within a Markov model is manually created. In some embodiments, a software package is utilized to create a “hidden” Markov model where one or more internal states are deduced by the software from multiple examples of input data with desired outputs. These hidden states follow the same Markov process of having state transition probabilities that are also determined by input data. The output of the hidden Markov model is a set of probabilities that apply to the external states (e.g., inspiration, exhalation, wait for inhalation). FIG. 14 depicts an exemplary hidden Markov model that receives an input matrix 190 of sensor data within a time window. The output of the model is a vector of probability of each state (e.g., stage of breath). The probabilities are independently calculated and may not sum to 100% in some embodiments.

Once a Markov model has been created (either manually or in a development tool) and vetted, it can be reduced to software code that is used within a breath detection device. The Markov model output can be calculated by the device processor periodically (e.g., every time step) to refresh the probability for each state. In practice, a new state is entered or an event is triggered when one or more related probabilities exceed a threshold (e.g., p(Insp)>90% means that inspiration has begun).

Breath Detection by State Machine

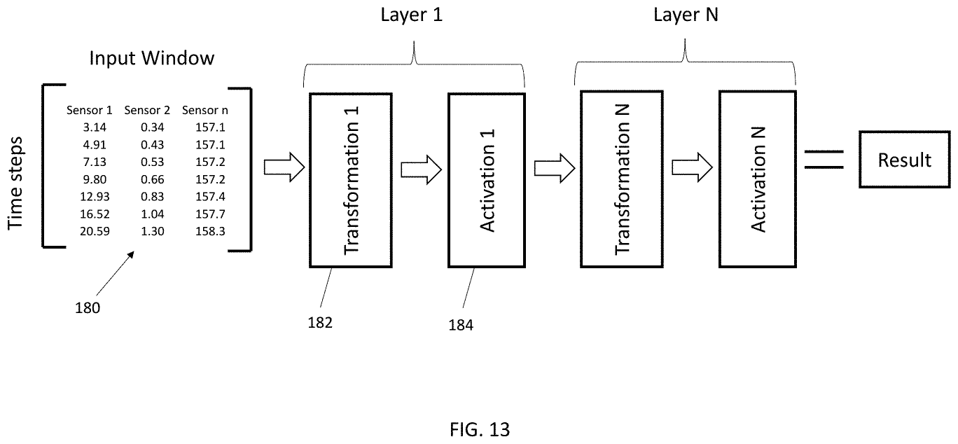

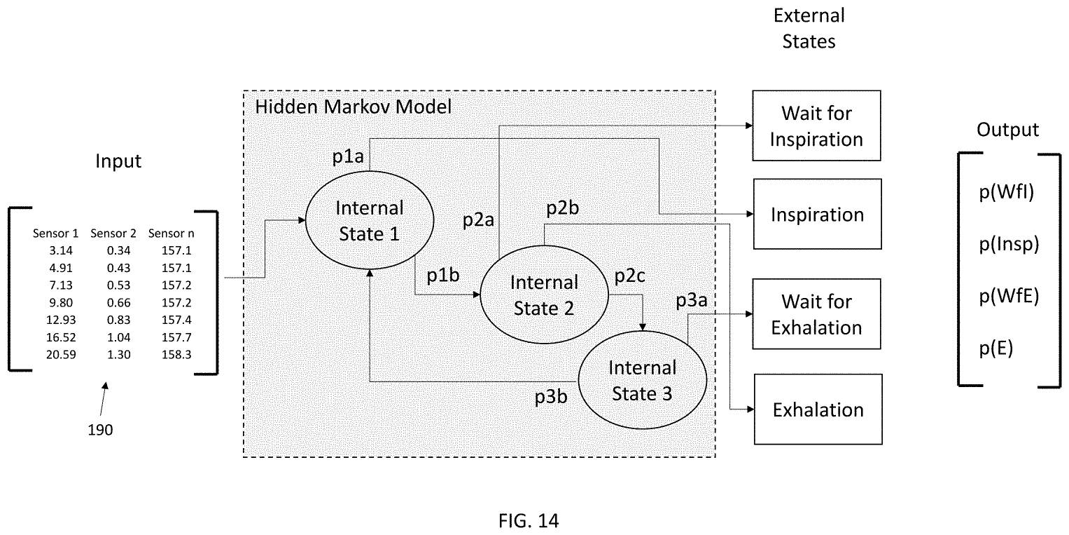

In some embodiments, a breath detection system utilizes state machine theory to improve breath detection accuracy. For example, a breath detection state machine steps through steps of the respiratory cycle, with entering and exit conditions for each state. Example states can be one or more of inspiration, exhalation and pause between breaths. FIG. 15A depicts an exemplary embodiment of a basic breath detection state machine. Not shown is that the system can exit to shut down from any state.

In some embodiments, a breath detection state machine tracks more discrete states within the respiratory cycle, such as inspiratory ramp, inspiratory peak, inspiratory slowdown, zero crossing, etc. Decision points (shown as diamonds in FIG. 15A) within a state machine can implement any of the analytical techniques presented herein to generate a decision.

Transition criteria are used to move between states. Example transition criteria are one or more of elapsed time within the state, pressure crossing a threshold, flow crossing a threshold, rate of pressure change with respect to time crossing a threshold, mean pressure inflecting, etc. Additional examples of state exit conditions depend on one or more of real time data, historical data and the mathematical functions thereof (e.g., slope, integral, mean, 2nd derivative, etc.).

In the event of state time-out (time limit exceeded), in one embodiment the system returns to a recovery state where it can utilize other sensed information to detect the current state or simply wait for the entrance criteria for an initial state in the sequence (e.g., inspiration).

The following table provides an exemplary state table for one exemplary embodiment. It should be noted that other embodiments begin looking for an exhalation event, or other type of respiratory event after exiting the Start-Up Initialize state.

| State | Description | Entry | Exit |
|---|---|---|---|
| Start-Up | System sets software | Off state | Successful completion of |
| Initialize | variables, zeroes sensors | | initialization tasks then |
| (state 200) | | | transfer to Wait for |
| | | | Inhale state |
| Wait for Inhale | System awaits | Start-Up Initialization | Change to Wait for Exhale |
| (step 202) | conditions that are | complete | state upon detection of |
| | indicative of inhalation | Reset complete | inhale event. |
| | | Exhale detected state | Error condition exits to |
| | | complete | Reset state |
| | | | Timeout exits to Reset |
| | | | state |
| Wait for Exhale | System awaits | Inhale detected in Wait | Change to Wait for Inhale |
| (step 204) | conditions that are | for Inhale state. | state upon detection of |
| | indicative of exhalation | | exhale event |
| | | | Error condition exits to |
| | | | Reset state |
| | | | Timeout exits to Reset |
| | | | state |
| Reset | System sets software | Time out or Error in | Completion of reset tasks |
| (step 206) | variables | another state. | then transfer to Wait for |
| | | | Inhale state |

FIG. 15B depicts an exemplary graph showing breath signal and state machine steps. Inspiration is marked when the respiratory signal crosses a threshold. In some embodiments, the end of inhalation is detected by a signal crossing a threshold, a zero-crossing event, an inflection point in the pressure signal, a reversal of the flow direction, or assumed to coincide with the beginning of exhalation. The beginning of exhalation is marked when the respiratory signal crosses zero. The end of exhalation is marked when the respiratory signal crosses zero. In some embodiments, thresholds other than zero can be used to mark the beginning and end of exhalation.

When a breath detection system enters the Reset state, it will reset its parameters and wait for a recognizable event. It should be noted that the initial event detected may not be the onset of inspiration. For example, the Reset state could exit into the corresponding state for any recognizable respiratory event

There can be many states in between the primary states presented herein. Some of these states can be related to a system activity, rather than a patient activity/input. In some embodiments, the system begins monitoring for the beginning of exhalation a fixed length of time after inspiration is detected (e.g., 400 msec). In some embodiments, this time delay is to permit a signal to stabilize. In some embodiments, the amount of time that the system waits is related to the amount of time it takes for a valve (e.g., NO gas, or purge valve) to close. In some embodiments, the system begins monitoring for the beginning of exhalation a variable amount of time after the inspiration is detected based on the data feed (e.g., inferred respiratory rate).

Respiratory Event Detection by Reflex Agent

In some embodiments, breath detection could be controlled by a reflex agent. Reflex agents are artificial intelligence agents that make decisions for what action to perform (e.g. deliver drug or wait for breath) based on the current set of sensor data and condition-action rules. Condition-action rules are predefined conditions (sets of sensor states) that are directly associated with taking a particular action. In contrast to a state machine that tracks the current state of the device and must follow certain paths between states, a reflex agent determines actions from the current sensor readings. Other embodiments use agent-based methods that use utility functions or learned behaviors instead of condition-action rules to determine which actions should be taken. The decision frequency or clock cycle of a reflex agent is typically at least 10 to 100 times more frequent that than the frequency of the events to be detected. This provides sufficient time for a drug delivery system to respond to a detected event while the event is still happening.

In some embodiments, there are two condition-action rules, as shown in the table below.

| Sensor reading | Action |
|---|---|
| Flow ≥ T | Deliver Drug |
| Flow < T | Withhold Drug |

Where T=a threshold value

Real Time Method Adjustments

In some embodiments, the breath detection system adjusts its method during patient treatment. Adjustments to its method include one or more of changing breath model, changing algorithmic approach, changing parameters, changing or enabling filters, enabling additional data collection, and enabling additional data processing. These changes can be made in response to sensed changes in patient activity, patient status (e.g., sleep vs. awake), device status, and patient environment. In one embodiment, a device adopts a less computationally intensive approach (e.g. less data filtration) when processor temperature exceeds a threshold.

In some embodiments, patient activity is detected via accelerometer and the system increases the algorithm sensitivity when patient activity is low and decreases algorithm sensitivity when patient activity is high. In some embodiments, the breath detection system applies or removes filters to breath detection input data (e.g., pressure) in response to sensed patient activity (e.g. when the patient is talking, moving, or sleeping). In some embodiments, a talking filter is customized by the device based on the user's voice properties as they are transduced by the respiratory sensor. In some embodiments, this adjustment in data filters is made based on time of day. In some embodiments, sleep is detected by one or more of heart rate, brain activity measurements, changes in body temperature, or activity level. In some embodiments, patient activity can detected by at least one of a gyroscope, a compasses, and GPS.

A breath detection system can also make adjustments to approaches and settings based on the condition of the breath detection system or overall device. In some embodiments, adjustments are made in response to detection of one or more of a partially or totally obstructed delivery and/or breath detection path (loss of signal), and an O2 concentrator delivering a pulse asynchronous with the respiratory cycle. In some embodiments, a breath detection system can select a more power efficient data filtration approach when device battery power falls below a threshold.

When a potentially problematic condition is detected, the breath detection system may do one or more of the following: generate an alarm condition, change detection sensitivity, alter signal processing settings, or alter drug delivery settings.

Event Confirmation (False Detection)

In some embodiments, a breath detection system analyzes additional data beyond the respiratory event to confirm an accurate event detection. In some embodiments, this involves analyzing the data occurring in the breath detection data channel after the event has been detected. In some embodiments, after an inhale is detected and the system switches to Wait for Exhale state, if an exhalation is not detected (i.e., times out), the system can infer that the detected inhalation event (i.e., a false positive) was not a true inhalation. Given that the system delivered drug to a falsely identified inspiratory event, the breath detection system can notify the dosing algorithm of this error so that it can make adjustments to future dosing. For example, a NO delivery system could increase the amount of NO in one or more subsequent breaths if a current detected breath has been determined to be a false positive. In some embodiments, when a false positive inspiratory event is detected, the breath detection system decreases the sensitivity on the breath detection sensor.

When a system detects exhalation (e.g., zero crossing) without a corresponding inhalation, the system can infer that an inspiratory event was missed (i.e., false negative). In response, some embodiments increase the sensitivity of the breath detection sensor to improve the ability to detect the next inspiratory event. Other embodiments alter the inspiration detection criteria in response to detection of a false negative (e.g., lower the inspiration threshold).

Clinical Event Detection, Logging and Alarms

In some embodiments, other events that have diagnostic value can optionally be captured. These events may include patient conditions such as hyperventilation, erratic breathing, or cessation of breathing. The events may also include system diagnostic events such as sensor failures or inconsistent readings. In some embodiments, the system alters its behavior based on these situations (e.g., alarms, notifying 3rd parties, dosing changes). In some embodiments, these events are logged for clinician review. In some embodiments, a breath detection system can notify clinical and/or emergency response personnel when a potentially life-threatening patient condition is detected (e.g. absence of breath exceeds a time threshold, or hyperventilation exceeds a time threshold). These notifications can be via multiple communication channels including but not limited to audible, visual, telephone, internet, or wireless communication.

Breath Modeling

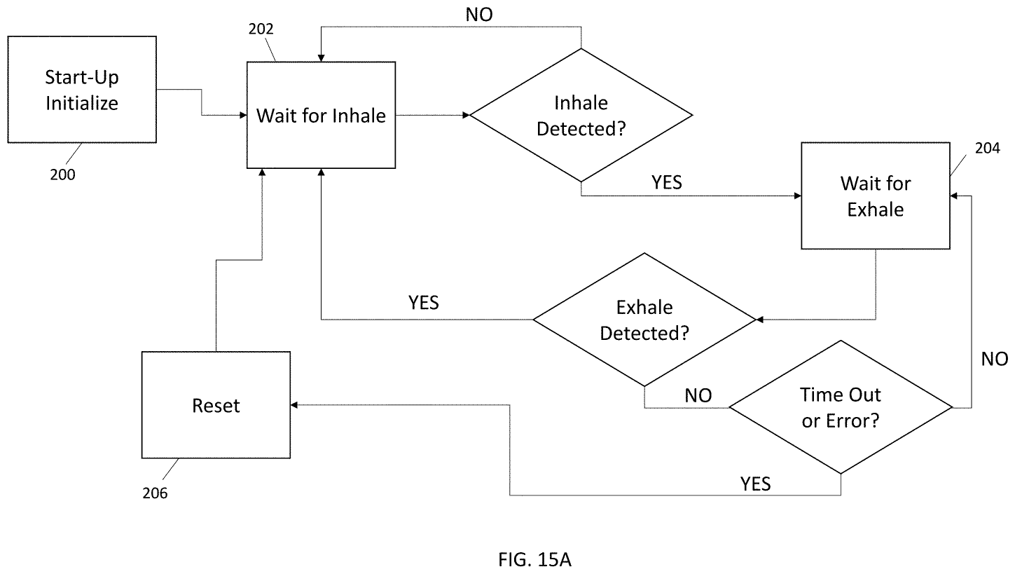

In some embodiments, the system assumes that the inspiratory flow profile has a particular shape (e.g., sinusoidal shape). In some embodiments, the system assumes a square-wave or trapezoidal profile to the breath waveform. In some embodiments, the system assumes a waveform that is consistent with a disease state. FIG. 16 depicts an exemplary graph showing representative breath profiles for various disease states. In some embodiments, the system assumes an inspiratory flow profile based on an indicated disease state that has been entered by the user or detected by the breath detection system. In some embodiments, the decision to select a particular flow profile shape is made by the system based on observations of one or more of the actual breathing cycle over time, the rate of change of the respiration sensor data, the breath period and other measurable breath characteristics. In one example, a breath detection system can observe that there is little to no pause between exhalation and inhalation, deduce that the patient has interstitial lung disease and apply the corresponding breath shape profile. In some embodiments, a profile is derived from observed data, either entirely or in combination with a pre-existing waveform, computational intensity and similarity to the target patient indication.

A breath profile can be used as input into an algorithm for adaptive filtering of one or more respiratory signals. In some embodiments, a band pass filter is utilized that focuses on the signal of interest. In some embodiments, the system changes the filter parameters based on properties of one or more of the respiratory signal, patient activity, environmental conditions (e.g. noise, barometric pressure), patient activity and type of concomitant therapies. Some more specific examples include tuning the filter parameters based on the state of the breath cycle, the time point within a breath that the patient is in, the amount of noise in the respiratory signal, the magnitude of the respiratory signal, or based on the model and/or sensor inputs. In some embodiments, other algorithm parameters are changed based on the perceived or entered breath profile for each disease. Exemplary parameters that could be changed include but are not limited to transition criteria between states, threshold values, and delays.

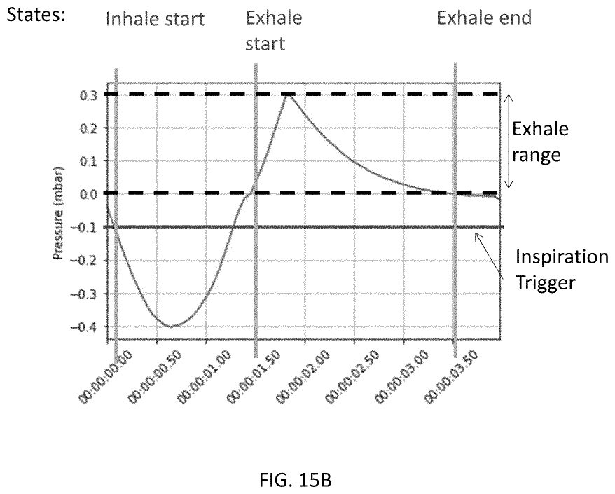

The duration of inspiration is a fraction of the breath period. Observation of multiple patient breath profiles and patients has revealed that the inspiration duration for a given breath period (or breath rate) is very repeatable over the range of breath rates for a given patient. FIG. 17 depicts an exemplary graph of a relationship between inspiratory duration (y axis) and breath period (x axis). In some embodiment, this relationship is entered into the breath detection system by a clinician. In other embodiments, the breath detection system measures this relationship for a specific patient utilizing known inspiration time points (e.g. entered by clinician or by utilizing an inspiratory flow sensor during patient characterization). In some embodiments, the relationship is linear across breath durations, with the inspiratory duration being 41% of the breath period. More complex regressions of this relationship could be used, but a linear approximation is usually sufficient for a given patient. In treating the patient depicted in FIG. 17, a drug delivery device would be able to utilize the equation and current breath period value to estimate the inspiratory duration. By knowing the inspiratory duration, the system can predict the optimal period and amount to dose the patient (e.g., first 60% of the breath). The optimal period varies between patients and by disease type and state.

In some embodiments, the relationship between inspiration duration and breath period can be determined for a given patient in the clinic prior to sending a patient home with the device. In some embodiments, the device utilizes the sensed timing of inspiration, beginning of exhalation and end of exhalation over a range of breaths to build a relationship between inspiration duration and breath duration for a specific patient. This may be accomplished with additional sensor inputs that are not available once the patient leaves the clinic. For example, the breath detection system may communicate with a inspiratory flow sensor via wired or wireless means during patient characterization. In some embodiments, the patient wears a mask and nasal cannula during characterization so that the breath detection system can capture data that relate inspiratory flow and inspiratory pressure as well as inhalation duration vs. breath period. In some embodiments, that patient undergoes multiple levels of exertion during breath characterization to provide multiple breath rates and breath periods in order to establish the breath period/inspiratory duration relationship.

In some embodiments, the system calculates a NO pulse width based on the expected inspiratory duration, as predicted by the breath rate. In some embodiments, the NO delivery system utilizes a mathematical relation and/or model between the breath period and inhalation period. In some embodiments, the NO pulse width is a function of the inhalation period. In one specific embodiment, the NO pulse width is a linear function of breath rate. In some embodiments, the parameters (e.g. coefficients, exponents, etc.) of the relation/model are updated each sequential breath. This enables a NO delivery system to adjust to the patient breathing patterns as the patient respirations change due to the patient environment, patient activity, delivery device interface with patient (e.g., nasal prong insertion level), and patient condition. In some embodiments, the system performs the calculations above for one or more breaths at the onset of treatment or after recovering from a fault before actual delivery of NO begins/resumes.

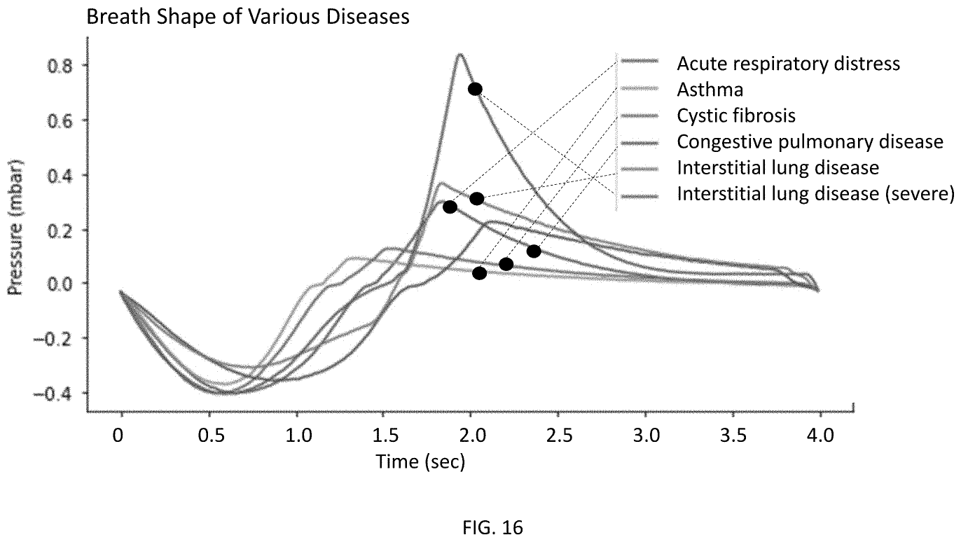

The system targets dosing a particular window within the inspiratory event. The dosing window is bracketed on the left by timing constraints stemming from the physics of detecting and delivering drug to a patient. The dosing window limit on the right is related to clinical considerations, such as the patient condition (i.e., not wanting to dose unhealthy lung in a COPD patient), and/or patient anatomical dead space volume. In some embodiments, the window is a fraction of the entire inspiration (e.g., 40% or 50%). FIG. 18 depicts an exemplary graph of a respiratory cycle with the initial 60% of volume of the breath targeted for NO dosing. The controller varies the amount of NO to be delivered during the dosing window by modulating one or more of the drug flow rate, drug concentration, and count of pulses within the treatment window (i.e. multiple short pulses rather than one longer pulse). In some embodiments, a NO delivery system sources NO from a tank with a fixed concentration. In order to dose a specific amount of NO in mg to the treatment window, the device delivers a combination of NO and purge gas to the treatment window. In some embodiments, NO and purge gas are mixed continuously to dilute the concentration of NO so that the flow rate of drug (i.e., transit time) remains unchanged but the NO delivery is spread out over a treatment window. In another embodiment, NO gas and a non-NO containing gas are delivered in alternating fashion to maintain a target flow rate while spreading NO delivery out over the target treatment window.

In some embodiments, the system calculates the difference between the optimum NO pulse duration for the prior breath and the actual NO pulse duration delivered to derive an error term. The error term may be represented in many ways including but not limited to a percent of the breath, a volume of gas, units of mass of gas, or units of time. Using the error term, the system reassesses the number of NO molecules that should be delivered in the next breath. Then, the system determines the NO pulse duration(s), concentration and flow rate and updates the parameters of the NO pulse function. In some embodiments, the inspiratory flow rate is known so that an actual volume of inspired gas can be determined. In other embodiments, the inspiratory flow rate is not known, and an inspiratory flow profile is assumed (e.g., sinusoid, trapezoid, square wave, disease-condition specific breath profile, etc.) when determining the NO pulse window error.

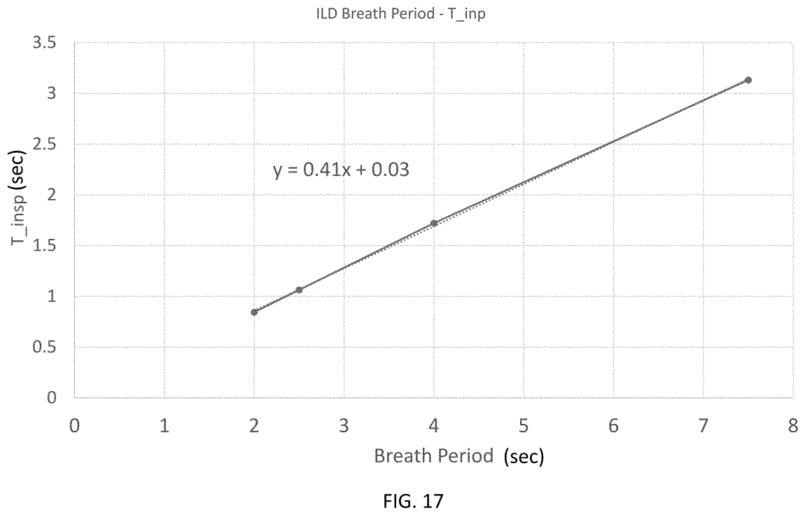

FIG. 19 depicts a graph of an example of using a trapezoidal model (line 210) of an inspiratory profile (line 212) to estimate the time point coinciding with an inspiratory volume limit (i.e., a point in the inspiratory volume beyond which dosing is not desired). The trapezoid is formed from the inspiration start time, inspiration end time. The actual height of the trapezoid is not required in order to calculate the time point coinciding with the target percent area of the trapezoid, which is the same time point for the inspiratory volume limit. The NO pulse shown in FIG. 19 did not last as long as it could have, leaving undosed volume within the target volume of the breath. Utilizing the time point from the trapezoidal model, the system can update the NO pulse duration to maximize dosing within the target window on a subsequent breath.

Patient Flow Characterization

As mentioned above, in some embodiments, the breathing profile for a given patient can be characterized at the onset of treatment and from time to time thereafter. One method utilized for patient characterization is to place a mask on the patient's nose and/or mouth over a cannula. The mask includes a flow sensor on the inlet/exit to quantify inspiratory and expiratory flows. As the patient breathes, the respiratory signal captured through the cannula can be correlated with total flow data from the mask by the system controller. This creates a mathematical relationship between total flow and the respiratory signal (e.g., cannula pressure in this example) that can be utilized for improving device performance. In one example, the breath detection system utilizes this relationship to inspiratory flow to improve the estimation of flow volume, which makes dosing more accurate. This flow information would also provide additional insight into the patient breathing profile which can be used to identify a patient disease type and degree. In some embodiments, flow information is utilized to improve a drug delivery device's ability to accurately dose a more accurate portion of an inspiration.

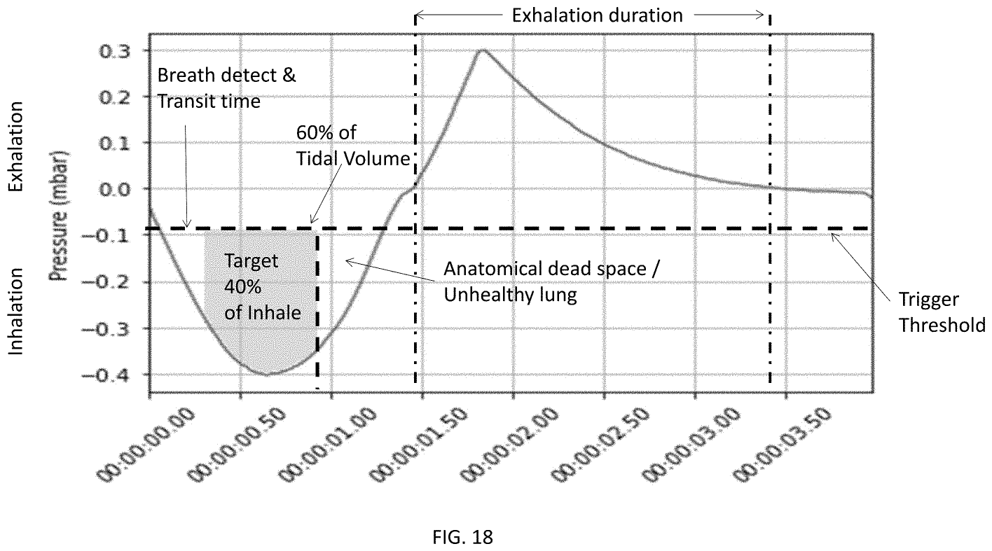

FIG. 20 depicts an exemplary system for characterizing patient inspiration. The patient wears a nasal cannula 220 that is pneumatically connected to a pressure sensor 222. The patient also wears a mask 224 that measures inspiratory and expiratory flow with one or more flow sensors 226. The signals from the pressure sensor 222 and flow sensor 226 are received by a controller 228 that creates a correlation between total flow, as measured by the flow sensor, and cannula pressure. The depicted example shows a nasal mask, however the same concept can be applied to a mask that covers both the mouth and nose. In some embodiments, a mask that covers the nose and mouth is utilized to characterize the cannula pressure signal when the patient breathes through the mouth.

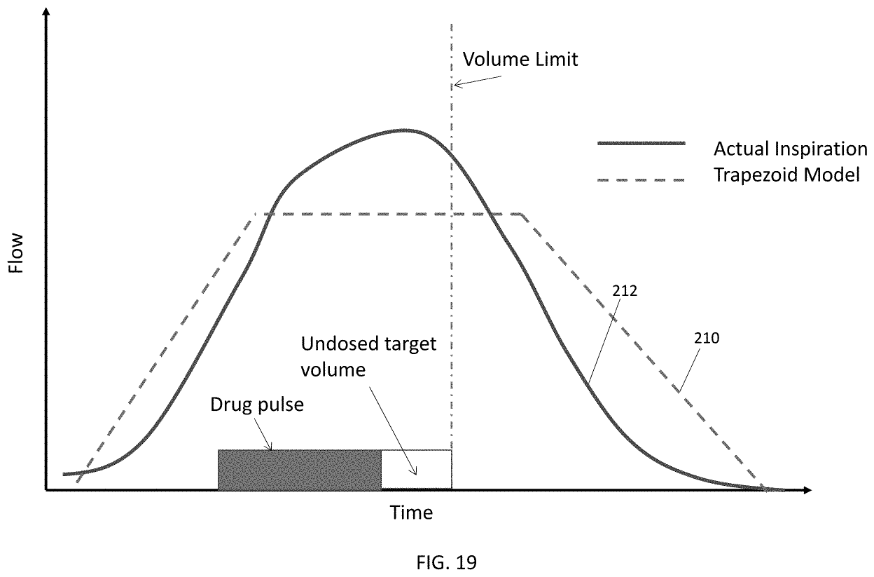

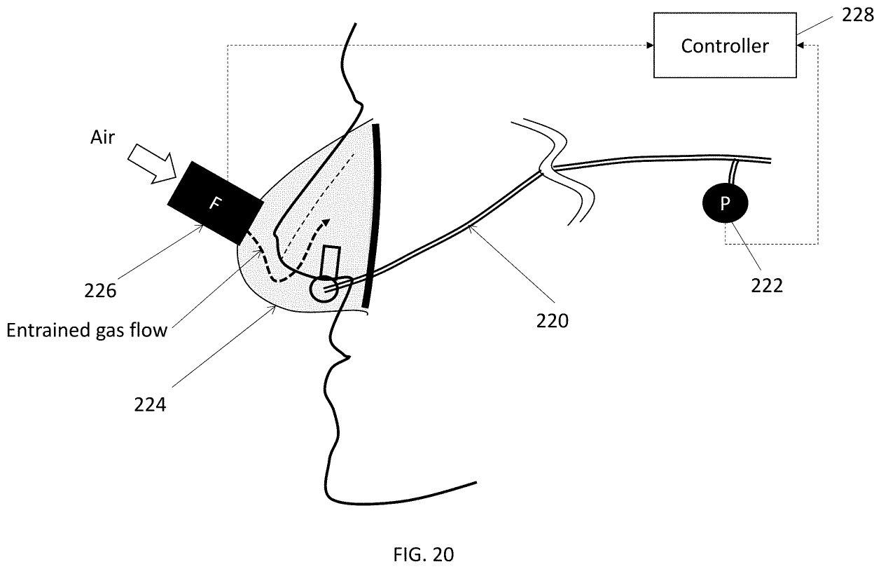

FIG. 21A depicts a graph of exemplary data from the collection of cannula pressure and mask flow data. The dashed line represents raw experimental data and the solid line represents a regression of the data into a mathematical function (e.g. polynomial, exponential, etc.) that characterizes the pressure/flow relationship. In some embodiments, this relationship is captured as a look-up table instead of a mathematical function. As the pressure within the cannula varies due to patient respiration, the controller can take pressure measurements at each time step and calculate the corresponding flow into and out of the patient using the equation.

FIG. 21B depicts an exemplary graph showing the relationship that the controller can utilize to calculate the volume of gas inspired as a function of the inspiratory flow rate. In some embodiments, the volume of inhaled gas is calculated incrementally at each time step by summing the mathematical product of the current inspiratory flow rate and the time step (Δt) duration. For example, if the instantaneous inspiratory flow rate is 101 μm and the time step is 0.1 sec ( 1/60 minutes), then the amount of gas inspired during the time step is 10 lpm times 1/60 min which equals ⅙ liters. These incremental volumes can be calculated by the controller in either real time or later to derive an estimate of inhaled volume. In some embodiments, this approach is utilized to estimate the tidal volume and the amount of inhaled volume in real time as the patient breathes.

In some embodiments, the tidal volume is tracked by the controller to record and analyze disease progression. For example, interstitial lung disease is a progressive disease where the tidal volume decreases over time. In one embodiment, a controller compares the average breath volume over a period time (e.g. 8 hours during sleeping, or a 24 hour period), at two different times, such as 1 month apart. If the two average breath volumes are similar, it is indicative that the disease has not progressed. If the current average breath volume is less than the prior average breath volume, the patient exhibits signs of disease progression. In some embodiments, if the breath volume change exceeds a specific amount or rate, the treatment device can do one or more of sound an alarm, record a message in its data files, and/or contact the patient's care givers. In other embodiments, measurement of tidal volume with and without therapy (e.g. nitric oxide) can enable quantification of treatment efficacy.

In another embodiment, a treatment controller utilizes inspiratory flow rate data to ensure that gaseous drug delivery flow rates are at or below the inspiratory flow rate. This ensures that all delivered drug is inhaled. In the event that a controller is unable to deliver sufficient moles of drug to a patient due to flow rate restrictions, some embodiments of a drug delivery device increase the concentration of the drug so that flow rates slower than the inspiratory flow rate can be utilized. In embodiments where inspiratory pressure can be measured during drug delivery, the controller can control the drug delivery rate to be less than the inspiratory flow rate in real time.

Performance Evaluation

The performance of a breath detection and dosing algorithm can be evaluated in many ways. Some embodiments of a breath detection system and drug delivery system can evaluate its performance in real time during patient treatment. In some embodiments, the amount of NO in moles introduced to the target inspiratory volume is utilized as a metric and compared against a target number of moles. For example, a dose of 20 ug per breath is targeted for the first ½ of a breath. A system that delivers bug to the first half of the breath would get a score of 50% (i.e., half the target dose). In some embodiments, NO delivered outside of the target range counts against the score (e.g., if 10% of the dose was outside the target window, the score is deducted 50%). In this example, weight of scoring for doses within the window vs. out of the window are equal. The weight of scoring within the target and outside the target can also be different. For example, in one embodiment the weight of score for being in the target is twice the weight of being outside the target. Thus, a dose that is delivered half into the target and half out of the target would get a score of 25% (50% for the on target dose minus 25% for the off target dose). In these examples, doses that are closer to target receive a higher total score.

In some embodiments, a breath detection and dosing scheme can be scored by the percentage of the inspiratory time that was dosed. In one example, it is desired to dose the entire duration of a 500 msec inspiratory event, however only 300 msec of the inspiration was dosed due to breath detection delay and pulse width settings. This breath detection and dosing scheme would receive a score of 60%.

A drug delivery system can utilize this scoring system as input to optimizing its treatment parameters over time to achieve one or more goals. Example goals include 1) placing at least 80% of the pulse within the target inspiration volume, 2) not placing any NO in a particular inspiration volume, 3) maximizing the percentage of NO within the target inspiratory volume, or 4) the percent coverage of the target inspiration duration in time. In one exemplary embodiment, a drug delivery system has a goal of delivering 80% of the pulse to the target inspiration volume. In the most recent n breaths, the system has delivered an average of 70% of the drug to the target inspiration volume. In response to this information, the system can alter the treatment with one or more of the following techniques: begin drug delivery earlier, increase the flow rate of drug (makes the pulse shorter for a given target mass of drug), and increase the concentration of drug (makes the pulse shorter for a given target mass of drug).

Zero Point and Compensation for Sensor Drift

The zero point for a respiratory sensor is a reference point or datum. The zero point for an algorithm may require periodic refreshing to account for one or more of changes in environmental conditions (e.g. altitude, temperature), changes in sensor performance (e.g. drift, aging, environmental conditions), and changes in patient conditions (e.g. respiratory rate, activity, cannula insertion, mask sealing, etc.).

In some embodiments, the respiratory sensor is a pressure sensor and the zero point is atmospheric pressure. The zero point can be measured through the delivery device when the patient is not breathing. The zero point can be measured when the delivery device is not connected. In some embodiments, a valve within the system exposes the respiratory sensor to atmospheric conditions for zeroing.

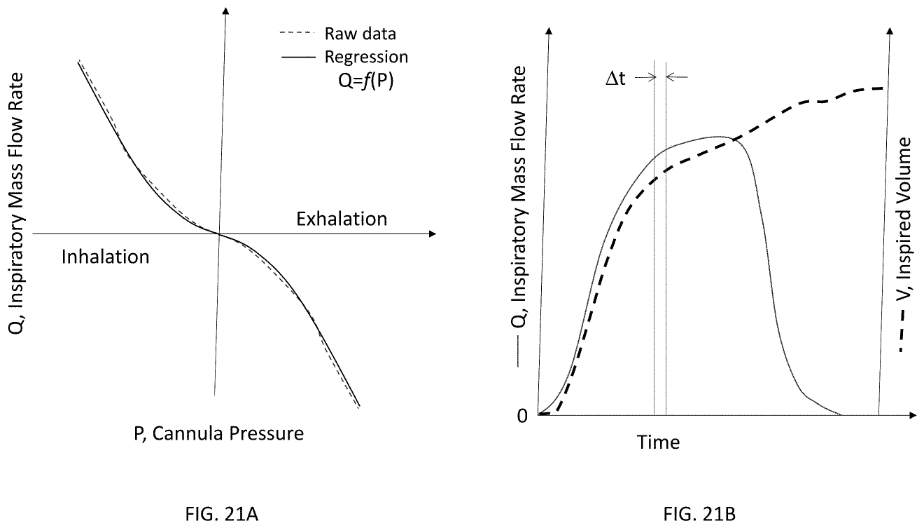

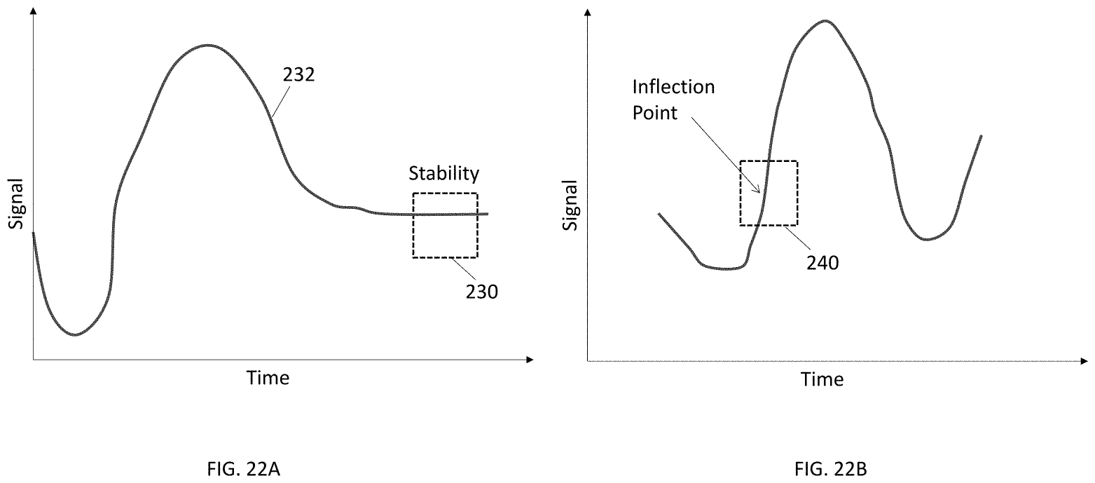

In some embodiments, the breath detection system looks for a stable period within the respiratory cycle and zeroes the sensor at that time. The stable period coincides with a period of no flow into or out of the patient, which could be at the end of inspiration or at the end of expiration. FIG. 22A depicts an exemplary graph showing an embodiment where the window 230 is located over a point of stability in the respiratory signal (curve 232).

In some embodiments, a breath detection system zeroes to the inflection point in the respiratory sensor data as the patient transitions from inspiration to exhalation (or vice versa). In some embodiments, the inflection point is detected within a window of respiratory signal values, as depicted in the exemplary graph shown in FIG. 22B. The window 240 is bounded by time (i.e., after peak inspiration/expiration, before a peak value, etc.). The vertical limits of the window provide a range of reasonable zero values to minimize the potential of mis-estimating due to noise.

In some embodiments, the zero point is determined in real time. In some embodiments, the zero point is determined with retrospective data analysis. The zero point does not need to be determined continuously or every breath, although some systems do this. It can be determined periodically, when the data are very clean and stable. Stability of the data can be determined by another statistical process within the breath detection system. Updating the zero point does not need to be performed in a large step function. In some embodiments, the system nudges the zero point in incremental steps in a direction determined by the zero point analysis.

Sensor drift may occur due to age, environment (temperature, pressure, humidity), and other factors. In addition, drift can occur based on patient factors, such as degree of insertion of the nasal prongs of a cannula. Ideally, selected sensors have built-in compensation for environmental factors. If not, compensation can be performed within software based on information from one or more additional sensors. For example, if a sensor is found to decay due to exposure to moisture so that it's reading drifts, a humidity sensor can provide data that can be used to adjust the reading to compensate for the expected drift. To characterize drift in a sensor, the breath detection system must provide the sensor with known inputs so that the sensor output can be measured, and a relationship or offset can be established. In some embodiments, the sensor is checked at one level for a simple offset. In some embodiments, a sweep of sensor outputs at multiple levels is compared to known input values so that a calibration curve or function can be derived. In one embodiment, a breath detection pressure sensor is exposed to atmospheric pressure to reestablish the zero point. In some embodiments, a drug delivery device sends known flows through a patient delivery device based on pump settings and the respiratory sensor is recalibrated based on those flow rates.

Breath Detection Sensor Redundancy

In some embodiments, a breath detection system utilizes two or more respiratory sensors of the same kind for redundancy and to detect sensor drift. In some embodiments, two sensors of different kinds are utilized so that their drift and failure modes differ. In one exemplary embodiment, a breath detection system utilizes a hot wire flow sensor and a delta-pressure flow sensor to measure inspiratory flow rate. Breath is detected when the average of flow rate between the two sensors exceeds a threshold (e.g. 0.5 lpm). When the difference between two sensor readings exceeds a specific threshold (e.g. 0.25 lpm), the system can generate an alarm. In some embodiments, the breath detection system operates by utilizing the average of the 2 or more respiratory measurements. In some embodiments, the speed of the two or more sensors differs. In some embodiments, the device makes respiratory event detection decisions based off the fast sensor but has the ability to flag a false positive if the slower sensor, at a later time point, does not also indicate that has an event has occurred. In some embodiments, a drug delivery device will abort drug delivery (e.g. stop the delivery of an individual bolus of drug) in the event that drug delivery has started and a false positive is announced at a later time point either sensor. In some embodiments, a breath detection system utilizes 3 respiratory sensors to measure the same measurand. When there is disagreement between the 3 sensors, the breath detection system continues operation, utilizing the two respiratory sensors that agree the most.

Safety Features

If the patient is not connected to a drug delivery device and the device provides a dose of drug, the device will be dosing the ambient air. This scenario can present risks to the patient (absence of treatment) but also to the environment by exposing nearby people to the drug and/or drug byproducts (e.g., nitrogen dioxide, nitrogen dioxide, etc.). In some embodiments, the system can detect when a patient is not connected and automatically stop dosing. A breath detection system can detect the absence of a patient by the absence of any detectable breath signal. Other embodiments of a breath detection system utilize an external device, patient worn sensors, or other sensors to detect the patient. In some embodiments, a temperature sensor within the proximal region of a nasal cannula is utilized to detect body heat to inform a drug delivery device that the delivery device is connected correctly (i.e. there is a temperature signal) and the cannula is installed (there is a temperature measurement from the patient's nose). Not all drug delivery devices cease treatment delivery when the drug delivery component is not connected. For example, O2 generators don't stop delivering oxygen in the absence of a patient because oxygen does not present a harm to the environment.

In some embodiments, a breath detection system can detect whether or not a patient is connected to the system and/or whether or not a delivery device is connected by observing the respiratory signal. If the respiratory signal is not changing, (i.e. a constant value, excluding noise, e.g. a pressure sensor only seeing ambient pressure), it is indicative of the patient not being connected. This could be applicable when a nasal cannula is not inserted into the nose, for example. In some embodiments, the system generates one or more of an audible, visual or tactile alarm in response to detecting that the patient is not connected.

In some embodiments, a NO delivery device can detect that the nasal cannula device is being worn by a patient by measuring the back-pressure to a pulse of gas (air, reactant gas, purge gas, product gas) through the delivery device. In some embodiments, the peak pressure for sending a known pulse profile (volume, flow rate) through the delivery device is characterized when the patient is not connected (i.e., delivering to atmospheric pressure). When the patient is connected to the delivery device, there is added back-pressure within the delivery device to push the gas into the patient as well. Similarly, delivering a bolus of gas to a mask that is sealed to the face of a patient requires higher back pressure than when the mask is not sealed to the patient. In some embodiments, when the back-pressure during pulse delivery falls below a threshold, the device alerts the user that the delivery device is not properly installed.

Dynamic Inspiratory Threshold

Many oxygen delivery devices have a fixed threshold for breath detection (i.e., indicating that inspiration has occurred when a respiratory signal crosses a threshold, such as 6 cm water). When a system uses a fixed threshold value for detection, it can be susceptible to false triggering due to noise in the sensor signal, patient activity, changes in the patient environment, activity of other devices, and other reasons. Hence, it can be beneficial to change the threshold from time to time to prevent false triggering (i.e. breath event detection) when a specific type of respiratory event is unlikely (e.g. inhalation is unlikely when the patient is exhalating). The threshold may also be changed due to a patient activity (e.g. when the patient is sleeping and breathing slowly, the threshold may need to be lowered to accurately detect inspiratory events). In some embodiments of an inhaled drug delivery device, the threshold for inspiration is dynamically updated throughout the patient treatment. This can compensate for varying patient and environmental conditions, such as nasal prong insertion level and wind levels, respectively.

Visiting FIG. 15B again, the range of respiratory signal magnitude during exhalation is noted. The range of exhalation values can provide an indication to the overall magnitude of sensor inputs. In other words, a large exhalation signal range would be indicative of a large inhalation range so that a more conservative, larger trigger threshold value can be used. Similarly, if the exhalation signal range is low, it is indicative of a weak respiratory signal and the system can respond by lowering the inspiratory signal threshold, increasing signal amplification, adjusting filters and the like to improve the odds of detecting inspiratory events.

Because the inhalation signal is often masked by the delivery of therapy (e.g., inhaled drugs), it can be useful to look at the quality of the respiratory signal during other parts of the respiratory cycle to determine the quality of the respiratory sensor signal. Measuring the quality of the signal is important because it is affected by many factors, including the patient's activity, mouth breathing and the position of the cannula relative to his nose. In some embodiments, the quality of the signal drives sensitivity, filtering and other compensations to improve signal quality although this may come at the expense of calculation time and/or detection speed. The signal during the end of inhalation, end of exhalation and other non-inhalation periods provide a time when the controller can collect respiratory sensor data that have not been masked or influenced by drug delivery. The controller can quantify the frequency and amplitude of noise in the signal during this time, for example, to inform breath detection methods and settings. In one embodiment, frequency content is determined by FFT analysis. For example, when a high level of noise (e.g. large peak at 40 Hz noise in the FFT results) is observed in the respiratory sensor signal, the controller opts to change the signal filter settings or type to remove 40 Hz content, thereby smoothing the respiratory signal and improving the confidence in detection of inspiration. In some embodiments, the controller adjusts threshold trigger values for more accurate event detection (i.e. rejection of noise). For example, the controller may increase the threshold value above the noise level to ensure that the noise does not generate a false respiratory event.

In some embodiments, the noise level can be estimated by observing the exhalation signal. This estimated noise level can also be used to set a floor for the detection threshold to prevent triggering from noise. In one illustrative example, a patient is utilizing a breath detection device while driving a car. The vibrations from the car introduce noise with an amplitude of 0.25V in a pressure-based breath detection signal. The system quantifies the amplitude of the noise during the quiescent period between exhalation and inhalation. The system then sets the inhalation detection threshold above the amplitude of the noise with some safety margin (e.g. 0.75V) to ensure that the environmental noise does not generate a false positive triggering event. Continuing with this same scenario, the patient opens the window in their car and the window noise increases the respiratory sensor noise signal to an amplitude of 0.75V. In response to this increase in ambient noise level, the breath detection system may respond by increasing the inspiratory threshold even further or by adopting another approach to breath detection. For the system to keep up with sudden environmental changes, some systems quantify environmental noise frequently (e.g. multiple times in the breath cycle).

It should be noted that the magnitude of the overall respiratory signal (expiration+inspiration range) can also serve as an input to determining the inspiration detection threshold in systems that have a dedicated lumen for breath detection or a respiratory sensor that is not affected by drug delivery through the delivery device. For example, a respiratory signal spans on average from 0.2V at the maximum expiratory flow rate up to 2.2V at maximum inspiratory flow rate, determined by the maximum and minimum values observed in a stream of data. The breath detection system defines the inspiratory threshold as 70% of the magnitude of the respiratory signal (0.7*(2.2V−0.2V)+0.2V=1.6V).

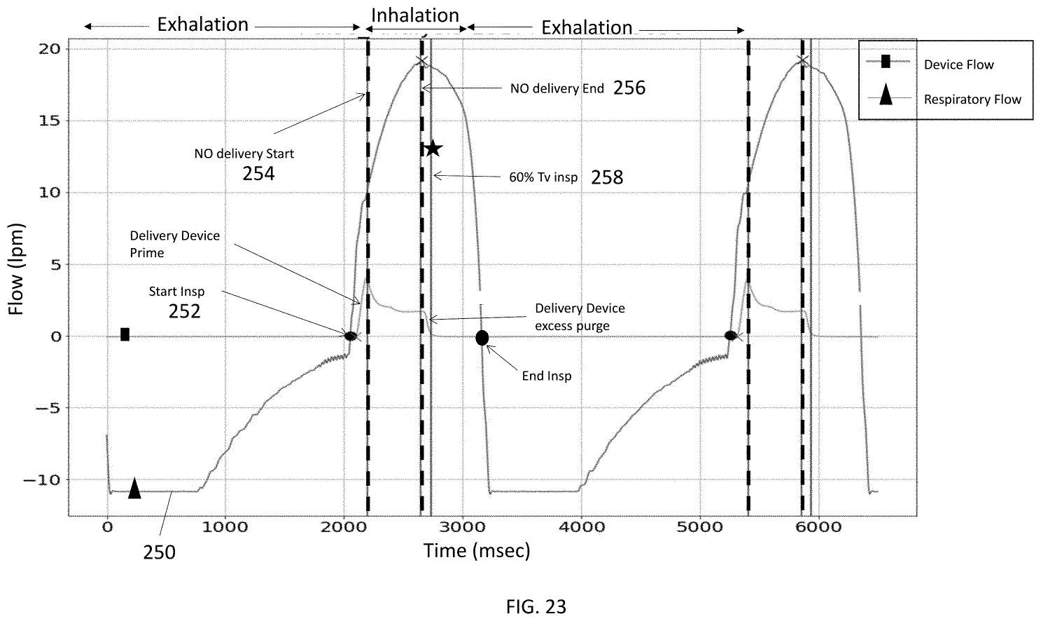

FIG. 23 depicts an exemplary graph showing a data related to a drug delivery system in operation with flow rates on the Y axis and time on the X axis. Positive flow is towards the patient, i.e., inspiration. The respiratory flow is shown as curve 250. The plot begins with the patient exhaling. When the respiratory flow crosses zero, the start of inspiration is shown at point 252. In this particular embodiment, the delivery system is filled with air between breaths. Thus, initial flow through the delivery system is to prime the delivery system with drug. Actual drug delivery out of the delivery device begins a short time later, on the order of tens of milliseconds, depending on the delivery device volume and the flow rate of the drug.

Initiation of drug delivery (i.e., drug entering the patient) is marked by a first vertical dashed line 254. Drug flow during priming can be faster than the flow rate during delivery to the patient to decrease the duration of the priming step. Once drug is exiting the delivery device, the flow rate is slowed to stretch out the delivery pulse to the desired duration. A second vertical dashed line 256 marks the end of the drug pulse delivery. The final drug molecules entering the patient are actually pushed by purge gas (e.g., air) so that the delivery device is devoid of drug between breaths. There is a brief additional flow of gas through the delivery device after drug delivery as a safety measure to ensure complete purging of the delivery device (i.e., the purge volume exceeds the drug flow path volume to ensure complete purging). A vertical line 258 to the right of the end of delivery dashed vertical line 256 denoted with a black star marks 60% of the volume of inspiration complete. The inspiratory event continues until the inspiratory flow crosses a threshold marking the end of inspiration. The difference between end of inspiration time and beginning of inspiration time is equal to the inspiratory duration. The difference between the time of an event in the respiratory signal in one breath and the time of that same event in a prior breath is equal to the breath period. The system can infer the duration of the drug delivery pulse for a subsequent breath as a function of these values. In some embodiments, the drug delivery pulse is calculated for every breath. In some embodiments, it is calculated for every nth breath, or when particular circumstances present themselves (e.g., the pulse no longer satisfies particular metrics).

### Example 1: Utilizing Measured Inspiratory Times

Inspiratory duration=(time of end of inspiration zero crossing for breath n)−(time of inhalation start of breath n)

Target drug delivery pulse coverage=C %

Drug pulse duration for breath n+1=C %*(Inspiratory Duration of breath n)

### Example 2: Inferring Inspiratory Duration from Patient Characterization

Breath Period=(time of inspiration n)−(time of inspiration n−1)

Inspiratory duration for breath n+1=m*(breath period)+b

Where m and b are the slope and offset, respectively, of the inspiratory duration to breath period relationship.

Drug Pulse duration for breath n+1=C %*Inspiratory Duration.

Carbon Dioxide Measurement for Breath Detection

In some embodiments, a drug delivery device monitors patient respiratory cycles by measuring carbon dioxide in a gas flow collected at the patient. In some embodiments, the sample flow is continuous resulting in an amount of inhaled drug in the gas sample. In some embodiments, the gas sample is introduced back into the reactant gas flow. Sample gas flow rates are typically in the 50 to 200 ml/min range. This is acceptable because the sample gas flow is much less than the inspiratory gas flow resulting in a negligible increase in inhaled CO2 concentration. In some embodiments, the gas sample is scrubbed for drug (e.g. NOx) before release into the atmosphere. In some embodiments, gas sampling flow is significantly reduced or turned off when drug is being delivered to prevent sampling of delivered drug which could lead to decreased delivered dose. In some embodiments, the carbon dioxide measurement is made at the patient (e.g. a nasal cannula prong assembly or mask with CO2 measurement capability).

In some embodiments, capnography technology is utilized to quantify CO2 levels in the air near the patient. In another embodiment, CO2 or NO2 levels are measured with a photoacoustic sensor. The gas sample collected at the patient typically passes through a dedicated sample lumen from the point of collection to the point of analysis. During inhalation, CO2 levels are atmospheric (roughly 420 ppb). When the patient exhales, the CO2 levels increase to roughly 35,000 to 50,000 ppm. The shift in CO2 concentration between exhalation and inhalation is rapid, providing a clear and fast indication of the beginning of inhalation. Furthermore, the presence of CO2 levels greater than ambient CO2 concentration is indicative of the delivery device being worn/installed correctly. Elevated CO2 levels on average can be determined with any speed of sensor, including slow sensors.

In some embodiments, the infrared light detector provides a signal to a processor within the system that post-processes the data for the detection of respiratory events. In one embodiment, the end of exhalation is determined when the CO2 level, as indicated by the infrared detector decreases from a higher level to below a threshold (e.g. 500 ppm).

In some embodiments, an inhaled drug delivery device marks the end of exhalation at the time that the CO2 pulse ends (i.e., CO2 concentration falls below a threshold, e.g. 20,000 ppm). In some embodiments, the device marks the end of exhalation when the rate of change of CO2 concentration becomes negative (i.e. declines and/or reverses). In some embodiments, the breath detection system enters a “wait for inhalation” state upon the end of the exhalation state. In some embodiments, the breath detection system changes the breath detection parameters and/or sensor sensitivity upon the end of the CO2 pulse associated with patient exhalation to improve detection of inspiratory events.

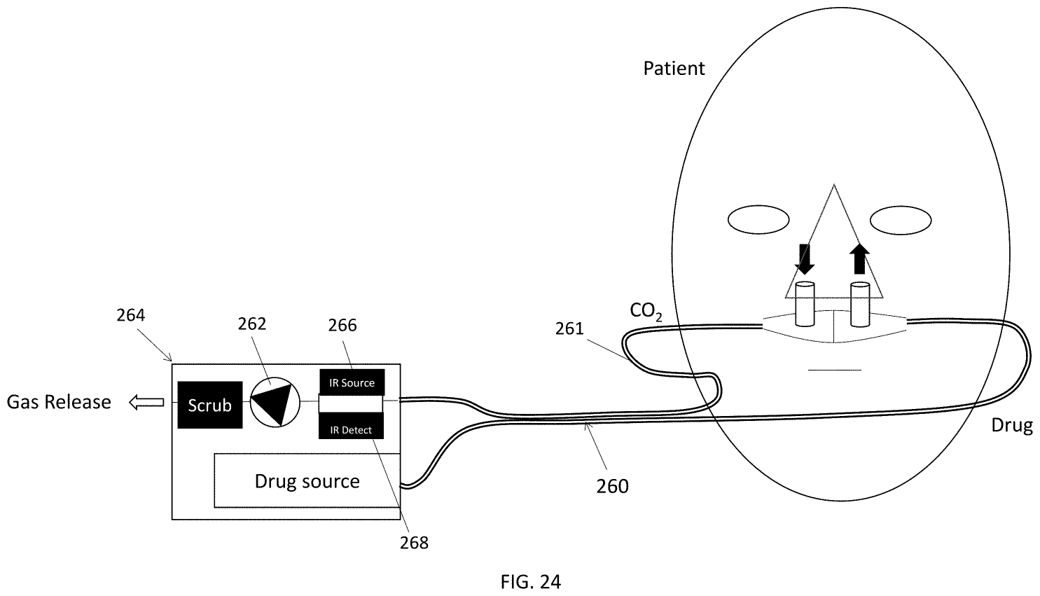

FIG. 24 depicts an embodiment of a breath detection system that utilizes CO2 measurement for respiration sensing. A patient is delivered an inhaled drug via a delivery device, such as a drug delivery lumen 260 (i.e., a cannula). A dedicated lumen 261 is utilized to pull a sample of the gas from a location near the patient. The sample gas flow rate and sample gas volume lumen are appropriately sized for acceptably fast response time. The gas is pulled by a pump 262 through a chamber 264. The chamber is typically a tube with a gas inlet and outlet, and a source and detector at opposite ends. The chamber may also be curved in shape, such as a waveguide to be small in size. At one end of the chamber, an infrared source 266 directs waves of infrared light through the gas (typically a wavelength of 4.26 μm for optimal CO2 absorption) to a detector. The detector 268 at the opposite end of the chamber measures how much infrared light passes through and therefore the breath detection system can calculate how much infrared light was absorbed by CO2 gas within the chamber. The signal analysis could happen within a processor in the sensor, the breath detection system, the NO generator, the drug delivery system or some other processor. The higher the concentration of CO2 gas, the less infrared light passes through due to absorption. The detector may be a nondispersive infrared (NDIR) type detector. The detector may be lead selenide, photo-detective, pyroelectric, thermopile or any other type known to those skilled in the art. An optical filter lens is used in front of the detector to notch filter or bandpass the light with a center wavelength of 4.26 um to remove other wavelengths. In some embodiments, the infrared source is pulse width modulated (PWM) to keep the detector from saturating. The infrared source may also be turned off during periods where the patient's exhalation is not being measured for CO2 concentration to conserve power. The more CO2 within the gas, the less infrared light is detected due to absorption. This relationship can be calibrated to provide a reliable measurement of the CO2 concentration of the gas along with using the ideal gas law for temperature and pressure changes. In some embodiments, the breath detection system utilizes a calibration gas that contains very low concentrations of CO2 that is either built into the system or sourced externally from time to time. In some embodiments, (not shown), the pump is before the measurement chamber. After measurement, the gas passes through an optional scrubber and is released into the atmosphere.

In some embodiments, the CO2 detector shown in FIG. 24 is removed from the drug source block and relocated to the mask shown in FIG. 20. This placement is in close proximity to the patient in order to achieve real-time measurement of the CO2 concentrations in the patient's exhalation breath. In some embodiments, this may be used by the breath detection system to decide when the drug source should be attempting to dose the patient. If the CO2 detector determines that the patient is exhaling, it may temporarily turn off the drug source to stop attempting to dose the patient. The CO2 detector may contain its own battery and be part of the physical mask worn by the patient. In some embodiments, the CO2 detector may use wireless communications, such as Bluetooth to communicate with the breath detection system.

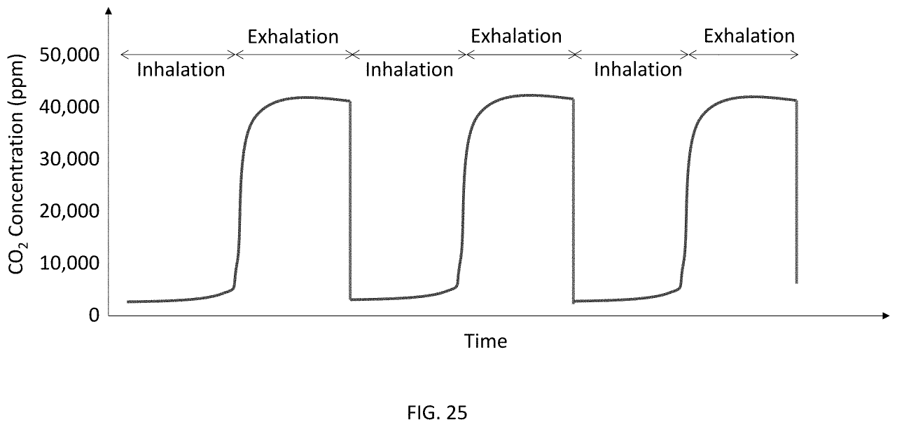

FIG. 25 depicts an exemplary graph of a data stream from a sensor measuring CO2 gas content near a patient. When the patient inspires atmospheric or oxygen-enriched air, the CO2 levels are low. When the patient exhales air from their lungs and alveolar spaces, the CO2 content in the sampled gas is high. A breath detection system can detect the beginning of inspiration and/or end of exhalation based on the magnitude and/or rate of change in the CO2 signal.

The transit time of a CO2 sample from the patient to the CO2 sensor may be too long to support real time breath detection decisions. In some embodiments, the CO2 concentration waveform is utilized retrospectively to confirm whether or not there was a breath. In the event that a breath detection system observes that a breath occurred based on CO2 measurements and that breath had not been detected and dosed with the primary method, a false negative can be recorded. In response to a false negative, one embodiment of a breath detection system can make adjustments to breath detection parameters (e.g. thresholds, amplification, filtration methods and levels). In another embodiment of a breath detection system, the dose of drug delivered is increased over one or more subsequent breaths to maintain a specific drug delivery run rate in response to a false negative breath detection event.

In some embodiments, CO2 or NO2 is measured at the patient as a respiration sensor. In one embodiment, a sensor is located in the nasal prong assembly. In some embodiments, the sensor is located in a mask.

Humidity Monitoring for Breath Detection

In some embodiments, the water content (i.e. humidity) of gas collected from the patient airstream or near the patient can serve as an indicator of respiratory state and can be used to detect respiratory events. This approach is generally applied to patients that are breathing atmospheric air without assistance (i.e. no ventilator, CPAP, etc.) In some embodiments, a breath detection system includes a humidity sensor located at or near the patient. In some embodiments, a breath detection system draws a sample of gas from near the patient through a dedicated lumen to a remotely located humidity sensor. As the patient inhales, the humidity of the gas is equal to or nearly equal to the ambient humidity. Exhaled gases, however, can have higher water content (e.g. 95% humidity at 37 deg C.). Hence, a controller can determine the timing of inspiration and exhalation by monitoring the humidity of a gas stream as measured by a humidity sensor.

Various types of humidity sensor can be utilized, including but not limited to an optical sensors (e.g. a lens that fogs with exhaled humidity), resistive humidity sensors, capacitive humidity sensors, and thermal humidity sensors. In some embodiments, a humidity-based respiration sensor system resembles the system in FIG. 24 with a humidity sensor substituted for the CO2 sensor.

Compensation for Coughing, Sneezing

Respiratory signals of actual inspiratory and exhalation events follow characteristic patterns. Artifact from coughing, sneezing and other non-respiratory events have different characteristic patterns. During operation, a breath detection system can qualify the characteristics of events in the respiratory signal. Events marked as inspirations for example, have the characteristics of inspiration and not the characteristics of a cough. More specifically, when a pressure sensor is used to detect respirations, inspirations cause pressure rates of change within a particular range. Coughs and sneezes cause pressure rates of change that are much higher. Hence, in one embodiment, a breath detection system can ignore events that have rates of change in the respiratory sensor signal that exceed the characteristics of the expected respiratory event.

In some embodiments, a breath detection system is able to detect coughing events (e.g. utilizes a correlation model) to identify a cough artifact in a respiratory signal. In some embodiments, after a cough has been detected, the breath detection system utilizes different criteria for detecting the next inspiratory event. For example, in one embodiment, the system is more conservative about when to start the next breath. In some embodiments, the system skips one or more breaths to wait for the return of a more normal breathing cycle. Normally, a breath detection system is required to trigger quickly and thus can be susceptible to interference or false positives. After a known cough or other aberrant event, some breath detection systems go into a mode where the system analyzes breath detection data for the end of coughing. Once the end of coughing has been determined, the system resumes normal breath analysis to search for the next new inspiratory event.

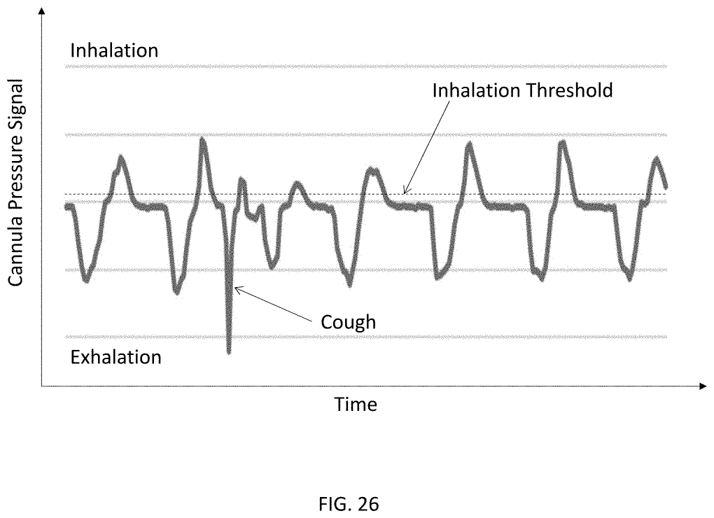

FIG. 26 depicts an exemplary graph showing a respiratory signal with a coughing event. The time history begins with the patient exhaling (signal going down and back to midline) then inhaling. After the second inhalation, the patient coughs. The cough is detectible by one or more of the following methods: The magnitude of the exhalation peak is significantly greater than previous exhalations, the rate of change for exhalation is greater during a cough than normal exhalation, the entire cough event is much shorter than a typical exhalation. When a cough is identified the system can keep a count of the coughs as a measure of patient health. The breath detection system can also respond to a cough by one or more of resetting the zero point, removing a portion of data before or after the cough from data analysis that spans many respiration cycles to remove the cough artifact.

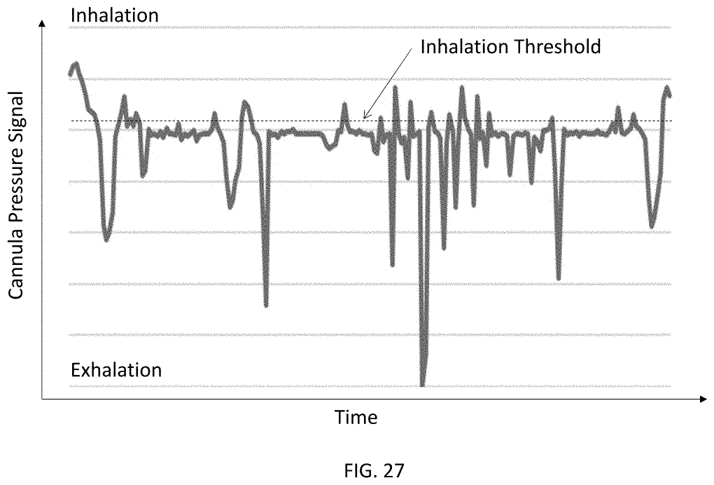

FIG. 27 depicts an exemplary graph showing a respiratory signal while a patient is talking. Talking is performed during exhalation, hence talking manifests as expiratory (negative) deviations in the respiratory signal. Inspirations are brief and aperiodic during talking. In one embodiment, a breath detection system detects that a patient is talking by observing frequent expiratory pulses in the data. Given that each patient has a characteristic acoustic frequency to their voice, some embodiments of a breath detection system are trained to recognize the frequency of the voice of a particular patient. One way of recognizing this voice frequency is by looking for a corresponding peak at the voice frequency in an FFT plot. In some embodiments, the breath detection system changes the inspiratory detection parameters in response to talking by lowering the inspiration detection threshold. This enables earlier detection to provide sufficient time for drug delivery. In some embodiments, a drug delivery device pauses drug delivery when the breath detection system detects the patient is talking. This can be acceptable for short periods of time.

Sensor Signal Isolation

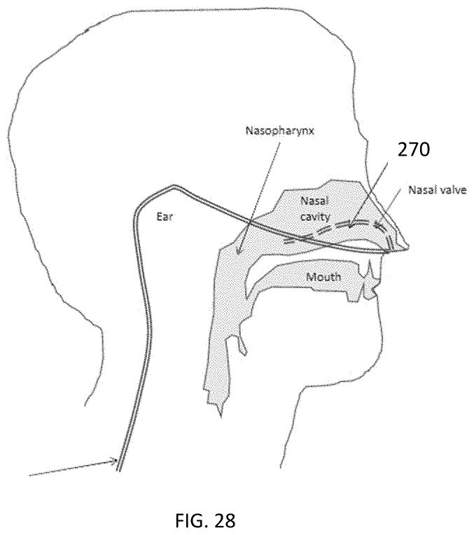

In some embodiments, as shown in FIG. 28, a cannula with long nasal prongs 270 (i.e., a cannula with prongs that reach more than the typical 15 mm into the nasal cavity and all the way to the posterior nasal cavity in some instances) is utilized to decrease interference in the breath detection signal from environmental noise and mouth breathing. In some embodiments, long nasal prongs are utilized with an intra-cannula pressure sensor. In some embodiments, nasal prongs extend into the patient beyond the soft palate or into the pharynx to enhance the ability to detect mouth breathing.

In some embodiments, a sensor is located on the nasal prong so that it is inserted into the patient. MEMS technology, for example, can be utilized to fabricate an insertable pressure sensor that can capture the pressure differences between inhalation and exhalation. Other measurement parameters have also been contemplated, including temperature and flow rate. By placing the sensor within the nasal sinus or deeper, the sensor is exposed less to environmental noise (e.g. open car window, jack hammer, other people talking). In some embodiments, the sensor is wired with wires attached to or embedded into the tubing of the cannula. In other embodiments, the sensor is wireless.

Disease Characterization and Progression Tracking

Many diseases affecting the lung and airway are progressive, meaning that the degree of tissue harm and volume of tissue affected will increase over time. In some clinical indications, healthy lung is recruited during inspiration before unhealthy lung. A pulsed drug delivery system (e.g. NO) can target specific regions of the lung by delivering drug during a specific portion of the inspired volume. For example, gas inhaled early during inhalation goes deep within the lung whereas gas inhaled late in inhalation doesn't get farther than the airway and bronchioles.

In some embodiments, a controller within an NO delivery device can automatically vary one or more NO pulse parameters (e.g. concentration, duration, delay, flow rate) and measure the effect on SpO2 values in the patient's blood. In some embodiments, the controller holds the quantity of NO constant but varies the NO pulse duration from a short duration (e.g. 10% of the inspiratory duration) to the entire inspiratory duration, holding the NO pulse duration constant at each incremental step for sufficient time for SpO2 to settle to a stable value. After sweeping through multiple pulse durations, the NO delivery system can begin delivering NO at the optimum duration, corresponding with the highest SpO2 measurements.

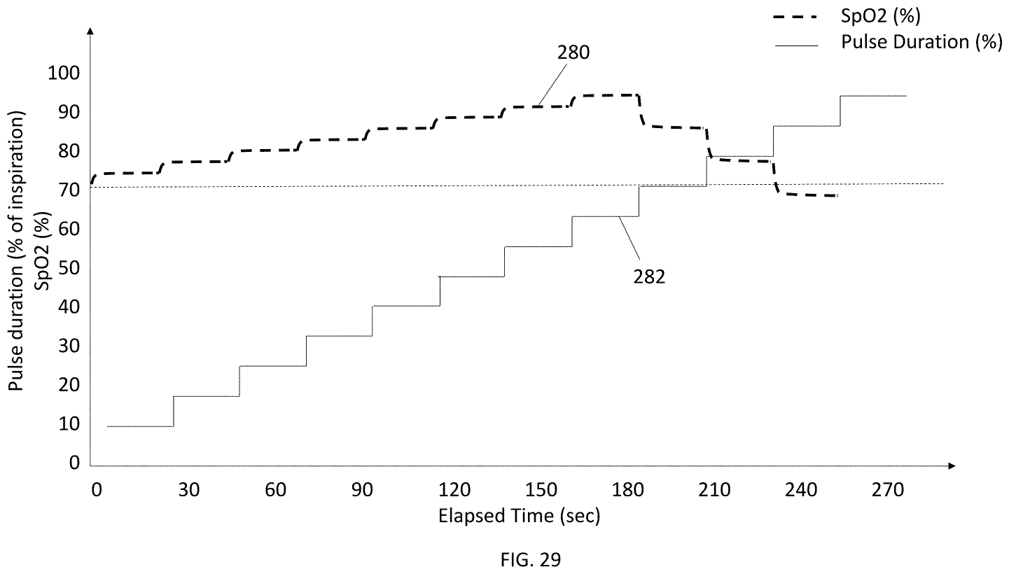

FIG. 29 depicts an exemplary graph of a pulse parameter sweep. In this example, the system sweeps pulse durations from 10% of the inspiratory duration to 100% of the inspiratory duration in 10% increments. Each level is held for multiple breaths to enable the SpO2 to settle. It can be seen that the SpO2 (curve 280) begins to decline for this exemplary patient when the pulse duration (curve 282) exceeds 70% of the inspiratory duration. In this case, the system will select a pulse duration that is less than 70% of the inspiratory duration for the next period of time. In other embodiments (not shown), the system increases pulse duration in units of time (e.g. msec) instead of inspiratory duration. It is preferred that the patient breathes at an even rate during characterization. It is also preferred that the patient is seated and resting during parameter evaluation to prevent confounding effects that could affect oxygen demand. In some embodiments, the system does not increase the SpO2 all the way to 100%. In some embodiments, the system stops increasing the pulse duration once the SpO2 begins to decline since this marks the transition from healthy lung recruitment to unhealthy lung recruitment. In some embodiments, the various parameter levels are evaluated in a descending order. In other embodiments, the various parameter levels are evaluated in a random order.

A similar type of approach can be performed for the quantity of NO delivered, the pulse delay and other parameters. The response of a patient to changes in NO pulse parameters and the optimum settings can be checked periodically (e.g. monthly) and used to track disease progression and ensure that optimal treatment is being delivered. Changes in optimal NO parameters over time can be indicative of current disease state and rate of progression which can also help inform care givers as they determine a treatment plan for a given patient.

Breath Detection Algorithm Optimization

Respiratory event detection algorithms are refined and tuned by subjecting them to exemplary data sets. In some embodiments, existing data are augmented to increase the number and variation of examples used for training and/or testing. Augmentations can include but are not limited to adding noise, increasing or decreasing signal amplitude, and generating synthetic data. One example method of generating synthetic data is using learned generative models such as Generative Adversarial Networks or Variational Autoencoders.

Prediction Modeling

As discussed previously, there may be a time delay from when the drug is produced by the source to the time that the dose is delivered to the patient. As can be seen for example in FIG. 24, the dose must travel through the drug delivery lumen to the patient. In a preferred embodiment, this time is very short (e.g. <100 ms), but in some cases may be longer. For example, for an ambulatory device, the drug delivery lumen may be very short because of the near proximity of the drug source to the patient. However, in other cases, for example such as ambulance, hospital, home-use or med-flight (air ambulance) cases, the time delay may be much longer due to the distance from the drug source to the patient. Therefore, some embodiments incorporate a prediction model algorithm to predict when one or more future patient inspirations will occur to overcome the time delay and to deliver the dose simultaneously to the inspiration period. In some embodiments, a prediction model may be used in concert with the breath detection algorithms discussed previously (e.g. utilizing both a breath detection algorithm and a prediction model and delivering drug in response to the first one that triggers). In some embodiments, the prediction model may use information about previous patient breaths, such as but not limited to inhalation detection thresholds, dynamic breath detection thresholds, transitions from different states, inspiration periods, exhalation periods, quiescent periods, peaks, troughs, respiratory sensor signal amplitudes, and respiratory sensor signal derivatives with respect to time. In some embodiments, the prediction model may use a breath profile algorithm to classify the patient's disease. As shown in FIG. 16, the breath shapes of various diseases differ from case to case. For example, if the prediction model classifies the patient as a case of asthma, the breath shape or profile will be different from that of a patient with severe interstitial lung disease. This information helps the prediction model to determine thresholds, either static or dynamic to determine the start of an inspiration period. In some embodiments, the classification determined by the prediction model may also be used to determine the N or the number of previous breaths used in the prediction of the beginning of an inhalation period. It can be appreciated that in some lung disease cases, having a larger value N may help the model more accurately predict the start of an inhalation period. In other cases, such as erratic breathing, may cause the model to use a lower value N or shut off the prediction model entirely and revert to solely using the breath detection algorithms described above. In cases where the patient breathes rapidly, their breath tends to be more periodic which can improve the accuracy of a prediction model. In some embodiments, the prediction model uses an activity profile in addition to the patient case classification. For example, if a person is determined to be sleeping, walking, walking upstairs or running, these activity profiles may have different lengths of time for inhalation, exhalation and quiescent periods. Breath rates will also be expected to be different for the different activity profiles. The following table presents how an exemplary model can interpret sensor information to determine patient activity.

Patient Activity Detection Logic

| Activity | Accelerometer | Altimeter | Breath rate |
|---|---|---|---|
| Sleeping | No activity | No change | Slow |
| Walking | Slow periodic motion | No/slow change | Medium |
| Walking | Slow periodic motion | Changing | Medium to Fast |
| upstairs | | | |
| Running | Fast periodic motion | No/slow change | Fast |

In some embodiments, the prediction model may work with one or more of the breath detection algorithms described above. For example, a breath detection model may be utilized to detect one event in the respiratory cycle in order to predict a subsequent event. In one specific example, the amount of quiescent time between exhalation and inhalation for a given patient has been characterized. The breath detection system is utilized to detect the end of exhalation. Then, the prediction algorithm is utilized to estimate the duration of the quiescent period so that drug delivery is initiated earlier in the inspiratory event than could be possible with a real time analysis approach. In other embodiments, owing to the amount of time required to generate and transfer drug from the drug source to the patient, a prediction algorithm may initiate drug generation and/or delivery before the inspiratory event with the expectation that drug will be delivered during the inspiratory event.

In some embodiments, the prediction model begins delivering the dose from the drug source so that it is ready for patient inspiration but must also determine when or when not to continue to deliver the dose. For example, the prediction model determines when to start producing the dose from the drug source and the drug travels through the drug delivery lumen to the patient, arriving at the precise time the patient inspiration occurs. The prediction model must also determine when to stop delivering the dose. For example, based on the patient clinical classification, the activity profile and/or the N samples used in the prediction, the model stops delivering the dose at the precise time the patient inspiration period ends. In some embodiments, the prediction model may intentionally end the drug dose at a predetermined time before the end of the inspiration period based on a classification or profile described above (e.g. to avoid dosing the latter portion of the breath for some clinical indications). In some embodiments, a drug delivery system will respond to a sensed inspiration event if it occurs prior to the predicted time (i.e. actual detected events override, or take higher priority to predicted events). In another embodiment, drug delivery initiated by a prediction can be terminated based on sensed termination of an inspiratory event.

In some embodiments, the prediction model may use a shifting technique or intentional delay to overcome random variations in patient inspirations. The shifting technique may apply a delay to the dose delivery based on a time parameter of a previous breath period, a running average of quiescent periods, a running average of inspiration trigger events and/or a running average of inspiration periods. In another embodiment, the shifting technique may alter the period of dosing based on a running average of previous breath inspirations. In some embodiments, the prediction model is able to remove noise or disruptions in the patient's respiratory signal. As shown in FIG. 4A, the signal conditioner may contain a filter to remove noise from the respiratory signal. As discussed previously, the filter may be low-pass or another type to remove patient speech, ambient noise such as street noise or any other type of noise contained in the respiratory signal. In some embodiments, the prediction model may use one or more additional sensors to determine a noise event. For example, the prediction model may use an accelerometer, microphone or motion sensor to detect a signal noise event and remove it from the measured respiratory signal. If the drug source is an ambulatory device, the accelerometer may detect a sharp movement such a drop or vibration due to transportation. The signal noise event is detected, and disruption is removed from the respiratory signal. In some embodiments, the prediction model may detect a respiratory signal rate of change that is above a threshold to identify a signal noise event, such as the patient coughing or sneezing and removes it from the respiratory signal.

In some embodiments, the prediction model may utilize a variety of sensor types including but not limited to a patient's electrocardiogram (ECG/EKG), transthoracic impedance and/or exhaled carbon dioxide. For example, the frontal QRS complex axis in the ECG/EKG signal varies with respiration as there is displacement of the heart muscle anatomically with diaphragmatic motion. In some embodiments, the prediction model uses this information for respiratory inhalation detection. T-wave inversion or variation in polarity may also occur during the respiratory cycle. Also, variations in the patient's transthoracic impedance may be indicative of changes in chest compression such as during coughing or sneezing. Furthermore, sensed CO2 in the dedicated sample return lumen may be indicative of the person coughing or sneezing. In one embodiment, the prediction model captures these noise events and removes them from the patient's respiratory signal to continue accurate dosing.

NO Generation/Delivery

The above systems and methods relating to breath detection can be used with systems and methods of nitric oxide (NO) generation and/or delivery for use in various applications, for example, inside a hospital room, in an emergency room, in a doctor's office, in a clinic, and outside a hospital setting as a portable or ambulatory device (e.g., in home). An NO generation and/or delivery system can take many forms, including but not limited to a device configured to work with an existing medical device that utilizes a product gas, a stand-alone (ambulatory) device, a module that can be integrated with an existing medical device, one or more types of cartridges that can perform various functions of the NO system, and an electronic NO tank. The NO generation system uses a reactant gas, including but not limited to ambient air, to produce a product gas that is enriched with NO.

An NO generation device can be used with any device that can utilize NO, including but not limited to a ventilator, an anesthesia device, a defibrillator, a ventricular assist device (VAD), a Continuous Positive Airway Pressure (CPAP) machine, a Bilevel Positive Airway Pressure (BiPAP) machine, a non-invasive positive pressure ventilator (NIPPV), a nasal cannula application, a nebulizer, an extracorporeal membrane oxygenation (ECMO), a bypass system, an automated CPR system, an oxygen delivery system, an oxygen concentrator, an oxygen generation system, and an automated external defibrillator AED, MRI, and a patient monitor. In addition, the destination for nitric oxide produced can be any type of delivery device associated with any medical device, including but not limited to a nasal cannula, a manual ventilation device, a face mask, inhaler, or any other delivery circuit. The NO generation capabilities can be integrated into any of these devices, or the devices can be used with an NO generation device as described herein.

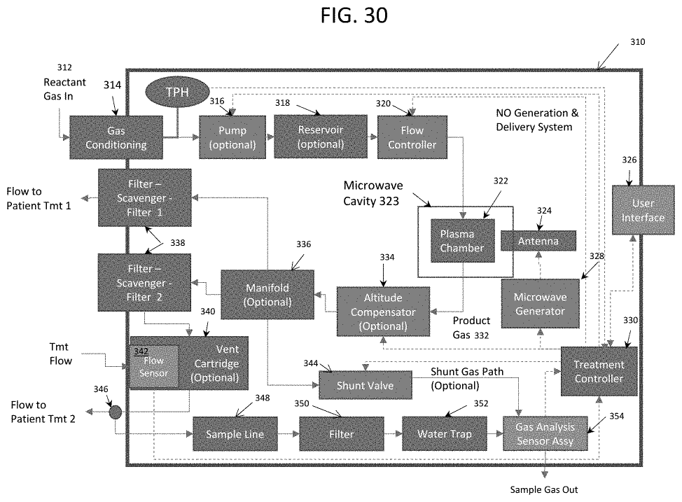

FIG. 30 illustrates an exemplary embodiment of a NO generation system 310 that includes components for reactant gas intake 312 and delivery to a plasma chamber 22. The NO generation system shown in FIG. 30 is an example of an NO system that can be used in conjunction with the disclosed breath detection systems for utilizing the information therefrom to deliver a drug (NO) to a patient based on detected respiratory events. Reactant gas enters the system through a gas conditioning cartridge 314 that includes one or more of a chemical scrubber (e.g. VOCs, ammonia), particulate filter, and one or more humidity adjustment mechanisms. A temperature, pressure and/or humidity (TPH) sensor can characterize the physical properties of the reactant gas. This information is transferred to the treatment controller for input into the NO generators calculation of microwave activity (e.g. frequency, power level) and reactant gas flow rate to achieve a target level of NO production. The system is configured to produce, with the use of a microwave generation circuit 328, microwave cavity 323, and one or more microwave source antennas 324, a product gas 332 containing a desired amount of NO from the reactant gas. The system includes a treatment controller 30 in electrical communication with the microwave generator 328 that is configured to control the concentration of NO in the product gas 332 using one or more control parameters relating to conditions within the system and/or conditions relating to a separate device for delivering the product gas to a patient and/or conditions relating to the patient receiving the product gas.

The controller 330 is also in communication with a user interface 326 that allows a user to interact with the system, enter a target NO dose level, view information about the system and NO production, and/or control parameters related to NO production.

The controller 330 is also in communication with a respiratory sensor. The respiratory sensor is in fluid communication with the inspiratory gas pathway and is utilized to capture respiratory data so that the controller can detect respiratory events.

In some embodiments, the NO system pneumatic path includes a pump pushing or pulling air through a manifold 336. In other embodiments, pressurized reactant gas is provided to the inlet of the NO generator. The manifold is configured with one or more valves (e.g., three-way valves, binary valves, check valves, and/or proportional orifices). The treatment controller 330 controls pump power, gas flow rate, the frequency of plasma pulses, the power in the plasma, and/or the direction of the gas flow post-electrical discharge. By configuring valves, the treatment controller can direct gas to the manual respiration pathway, the ventilator pathway, or the gas sensor chamber for direct measurement of NO, NO₂, and O₂ levels in the product gas. In some embodiments, respiratory gas (i.e., the treatment flow) can be directed through a ventilator cartridge that measures the mass flow of the respiratory gas and can merge the respiratory gas with NO product gas.

The output from the NO generation system in the form of the product gas 332 enriched with the NO produced in the plasma chamber 322 can either be directed to a respiratory or other device for NO delivery to a patient or can be directed to a plurality of components provided for self-test or calibration of the NO generation system. In some embodiments, the system collects gases to sample in two ways: 1) gases are collected from a patient inspiratory circuit near the patient and pass through a sample line 348, a filter 350, and a water trap 352, or 2) gases are shunted directly from the pneumatic circuit as they exit the plasma chamber 322. In some embodiments, product gases are shunted with a shunt valve 344 to the gas sensors after being scrubbed but before dilution into a patient airstream. In some embodiments (not shown), shunted product gases are diluted with air prior to exposure to gas sensors to provide NO/NO2/O2 concentrations in the range of the gas sensors. In some embodiments, product gases are collected from an inspiratory air stream near the device and/or within the device post-dilution. Within the gas analysis portion of the device, the product gas passes through one or more sensors to measure one or more of temperature, humidity, concentrations, pressure, and flow rate of various gasses therein.

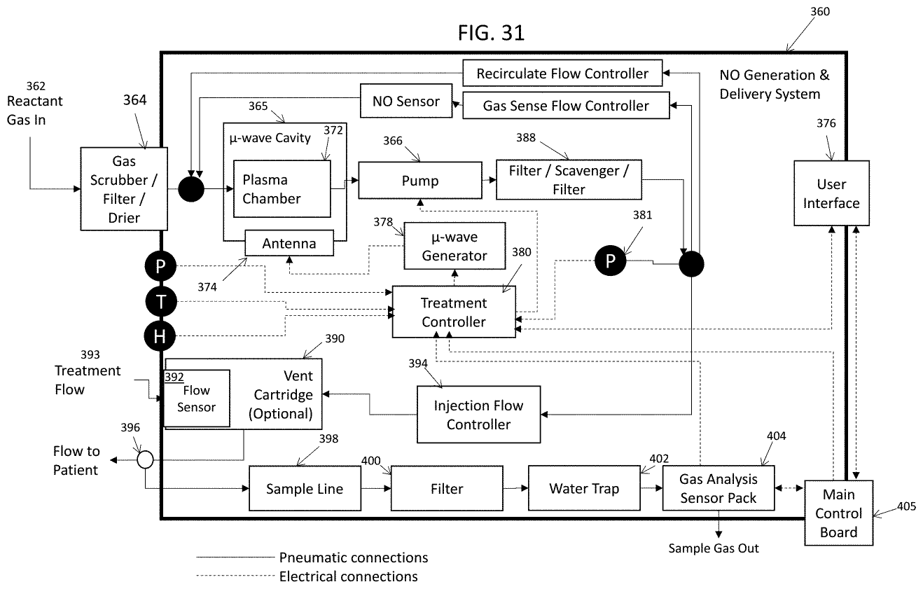

FIG. 31 depicts an embodiment of a NO generation and delivery system 360 with a recirculation architecture. Reactant gas 362 enters the system through a gas conditioner that includes one or more of a chemical scrubber, a particulate filter, and a humidifier/dehumidifier 364. Reactant gas passes through a junction where the recirculated gas enters the pathway and enters a plasma chamber. The plasma chamber is located within a microwave cavity 365 that forms a plasma ball within the reactant gas flow path. In other embodiments (not shown), NO is generated within the plasma chamber by electrical discharges between two or more electrodes. A pump 366 is used to pull gas through the plasma chamber. In some embodiments, the pump operates at a constant flow rate (e.g., 3 slpm). Gas enters the recirculation loop to make up for gas injected into the patient inspiratory pathway. Gas exiting the pump passes through a NO₂ scrubber to eliminate NO₂ formed by the NO generation process and NO₂ formed from oxidation of NO as it travels around the recirculation loop. The NO₂ scrubber includes one or more filters before and/or after it to prevent migration of scrubber material and capture particles released from the scrubber and potentially other parts of the system. After exiting the scrubber/filter stage 388, the gas enters a node that is maintained at constant pressure, as measured by pressure sensor 381. There are two or more flow paths exiting the constant pressure node. One flow path leads to a flow controller that controls product gas injection into the patient inspiratory stream. In some embodiments, constant concentration and pressure NO is introduced to the patient inspiratory stream in proportion to the inspiratory stream mass flow rate. The node is maintained at constant pressure to facilitate and improve the accuracy of the injection flow controller. A second flow path and flow controller can be utilized to maintain a target pressure at the node and return product gas to the beginning of the recirculation loop (pre-plasma chamber). A third and optional flow path includes a flow controller that regulates the product gas flow through one or more gas sensors used to monitor the product gas NO and/or NO₂ levels within the recirculation loop. A microwave antenna 374 within the resonant microwave cavity 372 is energized by a microwave generator 378 that produces microwaves of varying pulse frequency, pulse duration, and/or power level based on desired treatment conditions received from a treatment controller 380. A user interface 376 receives desired treatment conditions (dose, treatment mode, etc.) from the user and communicates them to the main control board 405. In some embodiments, the main control board 405 may include a controller, processor and/or other computer hardware/software for controlling other elements/components of the system, including other controllers. The main control board 405 relays to the treatment controller 380 a target dose and monitors measured NO concentrations from the gas analysis sensor pack 404. The main control board 405 monitors the system for error conditions and can generate alarms, as required.

Reactant gas 362 is converted into product gas when it passes through the plasma chamber 372 and is at least partially converted into nitric oxide and nitrogen dioxide. Ambient gas measurements made by pressure, temperature and/or humidity sensors on the left edge of the image are used as inputs to the treatment controller for plasma compensation to improve dose accuracy. In some embodiments, the treatment controller varies on or more of the duty cycle, the frequency and/or the power of the microwave pulses as humidity increases from 10% to 50%, for example, to maintain a constant production level of NO. In some embodiments, the treatment controller varies one or more of the microwave pulse duty cycle, pulse frequency and/or power as pressure increases in the plasma chamber to maintain a constant production level of NO. In some embodiments, the ventilator cartridge 390 includes a mass flow sensor 392 that measures the treatment gas flow 393. The treatment gas flow measurements from the flow sensor 392 serve as an input into a reactant gas flow controller via the treatment controller 380. In some embodiments, the inspiratory mass flow sensor is utilized as the respiratory sensor for breath detection. After product gas is introduced to the treatment flow, it passes through inspiratory tubing and optional humidifier (not shown) or moisture exchanger (not shown). Near the patient, a fitting 396 is used to pull a fraction of inspired gas from the inspiratory flow, through a sample line 398, filter 400, water trap 402 and humidity exchange tubing (e.g. Nafion) to prepare the gas sample and convey it to gas sensors 404. Gas sensors may measure one or more of nitrogen, oxygen, nitric oxide, nitrogen dioxide, helium, and carbon dioxide. Sample gas exits the gas analysis sensor pack 404 to ambient air. In some embodiments, the sample gas is scrubbed for one or more of NO, NO2, and medicines prior to release to the environment. In some embodiments, the system 360 can optionally direct gas through a shunt valve 394 and shunt gas path directly to the gas sensor pack and out of the system. In some embodiments involving the shunt valve 394, the manifold 386 includes a valve (not shown) to block flow to the filter-scavenger-filter when the shunt valve 394 is open.

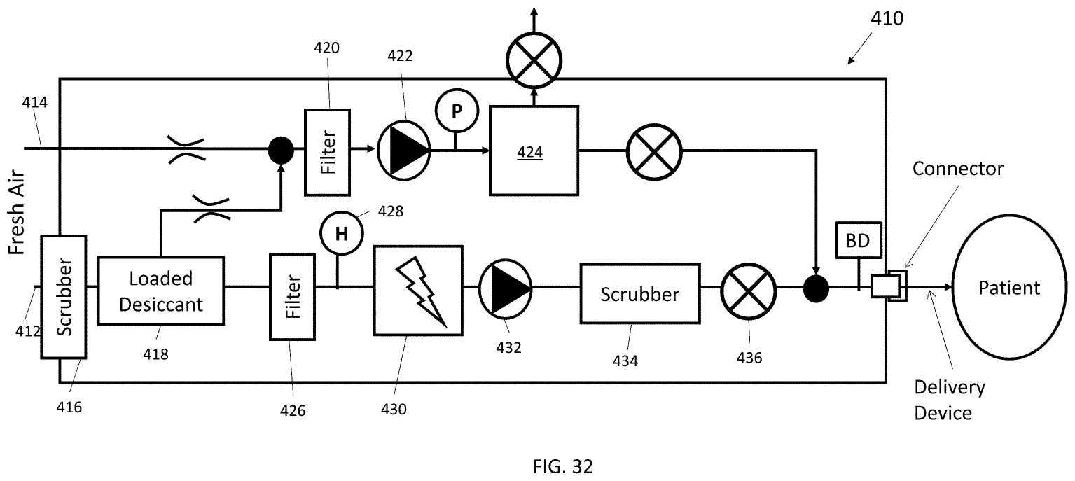

FIG. 32 depicts a pulsed NO delivery system 410 with a pressurized scrubber and pressurized bypass architecture. Reactant gas (i.e., air) enters the system into either a desiccant pathway 412 or a bypass pathway 414. Gas passing through the desiccant pathway is scrubbed for VOCs and harmful chemicals using a scrubber 416 prior to passing through desiccant 418 where it is either partially or completely dried. A portion of the dried reactant gas exits the drying component and is introduced to the bypass gas flow. The ratio of the blending is set in this embodiment by critical orifices, however other embodiments utilize proportional valves and/or pump speed to set the flow rate in each channel. In this embodiment, the mix ratio (desiccated vs. non-desiccated gas) is the same for all environmental conditions, resulting in some variation in purge gas humidity. The main objective of drying the purge gas (i.e. gas in the bypass channel) is to prevent condensation within the system when ambient conditions are humid. The mix ratio is selected to ensure that purge gas does not condense at worst case ambient humidity levels and purge gas pressures. Gas in the bypass pathway flows through a particle filter 420 and a pump 422 before it is accumulated in a purge reservoir 424, the exit of which is controlled by a valve. In the event that patient respirations are slow and too much gas accumulates in the bypass reservoir, a pressure relief valve can be opened based on the pressure within the bypass reservoir, as measured by a pressure sensor. Other embodiments use a permanently open orifice to continuously bleed pressure from the purge reservoir to prevent over-pressurization. Other embodiments use a proportional flow valve at the exit of the accumulator (not shown).

On the desiccated flow path, reactant gas passes through a particle filter 426 to remove particulate that could be altered in the plasma chamber or clog the flow path and/or components downstream. A humidity sensor 428 measures the humidity of the reactant gas to provide an indication when the desiccant stage has been exhausted. Reactant gas then flows into a plasma chamber 430 where nitric oxide is formed in the reactant gas due to elevated temperatures from making a plasma in the gas. The plasma is formed by an arc discharge between two electrodes in some embodiments. In other embodiments, the energy from microwave antennas is focused in a small area where a plasma ball forms. Product gas (reactant gas+NO) exits the plasma chamber and flows through a pump 432 that pressurizes a NO2 scrubber 434 with product gas. The exit of the pressurized scrubber is controlled by a valve 436. In some embodiments, the valve is a proportional valve.

The NO generation and delivery system depicted in FIG. 32 operates by delivering a pulse of NO followed by a pulse of purge gas. This approach leaves the delivery device devoid of NO-containing gas between breaths, thereby decreasing the amount of time that NO can oxidize into NO2. In some embodiments, the delivery device is a nasal cannula. In other embodiments, the delivery device is a tube connected to a mask. The delivery device connects to the generator with a connector. A sensor labeled “BD” in the figure measures pressure within the delivery device to detect breaths. In some embodiments, the BD sensor measures gas properties in the same lumen in which drug (NO) is delivered. In other embodiments, the BD sensor is in fluid communication with or located in a dedicated lumen for breath detection.

Not shown in FIG. 32 is the treatment controller that collects data from the various sensors and controls the plasma chamber, the pump and the valves to conduct the patient treatment. In some embodiments, the treatment controller is a hardware circuit. In other embodiments, the treatment controller is a software-driven microprocessor.

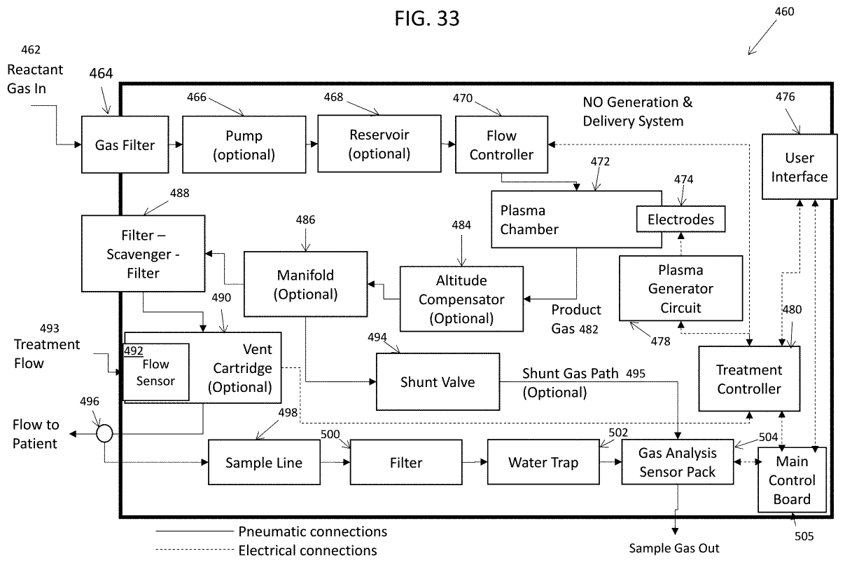

FIG. 33 depicts an embodiment of a NO generation and delivery system 460. Reactant gas 462 enters the system through a gas filter 464. A pump 466 is used to propel gas through the system. Whether or not a system includes a pump can depend on the pressure of the reactant gas supply. If reactant gas is pressurized, a pump may not be required. If reactant gas is at atmospheric pressure, a pump or other means to move reactant gas through the system is required. A reservoir 468 after the pump attenuates rapid changes in pressure and/or flow from a pump. Coupled with a flow controller 470, the reservoir, when pressurized, can enable a system to provide flow rates to the plasma chamber 472 that are greater than the pump 466 flow rate. This enables the use of a smaller, lighter, quieter and more efficient pump. Electrodes 474 within the plasma chamber 472 are energized by a plasma generation circuit 478 that produces high voltage inputs based on desired treatment conditions received from a treatment controller 480. A user interface 476 receives desired treatment conditions (dose, treatment mode, etc.) from the user and communicates them to the main control board 505. The main control board 505 relays to the treatment controller 480 the target dose and monitors measured NO concentrations from the gas analysis sensor pack 504. The main control board 505 monitors the system for error conditions and generates alarms, as required. The reactant gas 462 is converted into product gas 482 when it interacts with the plasma as it passes through the plasma chamber 472 and is partially converted into nitric oxide and nitrogen dioxide. An altitude compensator 484, typically consisting of one or more valves (for example, proportional, binary, 3-way), is optionally used to provide a back-pressure within the plasma chamber 472 for additional controls in nitric oxide production. Product gases pass through a manifold 486, as needed, to reach a filter-scavenger-filter 488 assembly that removes nitrogen dioxide from the product gas. From the filter-scavenger-filter 488, product gas is introduced to a patient treatment flow directly, or indirectly through a vent cartridge 490. In some embodiments, the vent cartridge 490 includes a flow sensor 492 that measures the treatment flow 493. The treatment flow measurements from the flow sensor 492 serve as an input into the reactant gas flow controller 470 via the treatment controller 480. After product gas 482 is introduced to the treatment flow, it passes through inspiratory tubing. Near the patient, a fitting 496 is used to pull a fraction of inspired gas from the inspiratory flow, through a sample line 498, filter 500, water trap 502 and Nafion tubing to prepare the gas sample and convey it to gas sensors 504. Sample gas exits the gas analysis sensor pack 504 to ambient air. In some embodiments, the system 460 can optionally direct gas through a shunt valve 494 and shunt gas path 495 directly to the gas sensor pack and out of the system. In some embodiments involving the shunt valve 494, the manifold 486 includes a valve (not shown) to block flow to the filter-scavenger-filter when the shunt valve 494 is open.

All patents, patent applications, and published references cited herein are hereby incorporated by reference in their entirety. It will be appreciated that several of the above-disclosed and other features and functions, or alternatives thereof, may be desirably combined into many other different systems or application. Various alternatives, modifications, variations, or improvements therein may be subsequently made by those skilled in the art.

What is claimed is:

1. A breath detection system, comprising:
one or more sensors configured to monitor at least one of one or more patient parameters and one or more environmental conditions relating to respiration;
a processor in communication with the one or more sensors, the processor configured to analyze information from the one or more sensors; and
determine an occurrence of at least one respiratory event using a threshold for detecting the at least one respiratory event, the threshold being variable throughout at least one respiration cycle to decrease the potential for a false determination of at least one subsequent respiratory event.

2. The breath detection system of claim 1, wherein the environmental conditions include at least one of ambient pressure, sound level and carbon dioxide levels.

3. The breath detection system of claim 1, wherein the processor is configured to vary the threshold using the information from the one or more sensors.

4. The breath detection system of claim 1, wherein the processor is configured to vary the threshold based on an elapsed time with respect to a current detected respiratory event.

5. The breath detection system of claim 4, wherein the elapsed time being based on the timing of prior respiratory events.

6. The breath detection system of claim 4, where the elapsed time relates to a rate of breathing.

7. The breath detection system of claim 4, where the elapsed time relates to a duration of inhalation.

8. The breath detection system of claim 1, wherein the processor is configured to vary the threshold based on a current respiratory rate.

9. The breath detection system of claim 8, wherein the processor is configured to vary the threshold for a period of time after a detected respiratory event, an amount of time of the variation of the threshold being based on the current respiratory rate.

10. The breath detection system of claim 1, wherein the processor is configured to vary the threshold based on a duration of a prior inhalation.

11. The breath detection system of claim 10, wherein the processor is configured to vary the threshold based on the duration of a drug delivery pulse.

12. The breath detection system of claim 1, wherein the processor is configured to vary the threshold based on environmental conditions communicated to the processor using one or more environmental sensors.

13. The breath detection system of claim 1, wherein the processor is configured to vary the threshold based on patient activity communicated to the processor using a patient motion detector such that the processor is configured to vary the threshold based on a detected increase or decrease in patient activity.

14. The breath detection system of claim 13, wherein the patient motion detector is an accelerometer.

15. A breath detection system, comprising:
one or more sensors configured to monitor at least one of one or more patient parameters and one or more environmental conditions relating to respiration;
a processor in communication with the one or more sensors, the processor configured to analyze information from the one or more sensors; and
determine an occurrence of a respiratory event requiring drug delivery, the respiratory event requiring drug delivery if measurements associated with the respiratory event equal or cross a threshold value for detecting the respiratory event, the threshold value being variable.

16. The breath detection system of claim 15, wherein the drug delivery comprises at least one dose of nitric oxide (NO).

17. The breath detection system of claim 16, further comprising a NO generation system configured to produce the NO for the drug delivery.

18. A method comprising:
monitoring, by at least one processor, a respiration associated with a patient, the monitoring of the respiration comprising receiving at least one respiratory measurement value measured by at least one sensor of a breath detection system;
determining, by the at least one processor, a respiratory event requiring drug delivery based at least in part on the at least one respiratory measurement value, the respiratory event requiring drug delivery if measurements associated with the respiratory event equal or cross a threshold value; and
varying, by the at least one processor, the threshold value associated with detection of the respiratory event based on conditions associated with at least one of patient parameters, environmental conditions, a respiration state, and respiration parameters.

19. The method of claim 18, a drug for the drug delivery is nitric oxide (NO).

20. The method of claim 19, further comprising, communicating, by the at least one processor, with a further processor associated with a nitric oxide (NO) generation system for providing NO, a timing of the delivery of NO from the NO generation system relating to the detection of the respiratory event.

21. The method of claim 19, further comprising, instructing, by the at least one processor, a nitric oxide (NO) generation system to generate and deliver NO based on the determined respiratory event.

22. The method of claim 18, further comprising determining, by the at least one processor, timing of changing the variable threshold based at least in part on a prior respiratory duration.

23. The method of claim 18, wherein the measurement value crosses a threshold for a minimum amount of time before recording the respiratory event.

24. The method of claim 18, wherein varying the threshold value comprises varying a magnitude of the threshold value.

25. The method of claim 18, further comprising determining, by the at least one processor, timing of changing of the variable threshold based at least in part on one or more of the timing and duration of a drug delivery pulse.
